(19) United States
(12) Patent Application Publication
(10) Pub. No.: US 2018/0333440 A1
(43) Pub. Date: Nov. 22, 2018

(54) BACTERIAL COMPOSITIONS AND METHODS OF USE THEREOF

(71) Applicant: THE UNIVERSITY OF BRITISH COLUMBIA, Vancouver (CA)

(72) Inventors: Brett FINLAY, Vancouver (CA); Marie-Claire ARRIETA, Calgary (CA); Shannon RUSSELL, Vancouver (CA); Stuart TURVEY, Vancouver (CA); Leah STIEMSMA, Vancouver (CA)

(21) Appl. No.: 16/051,844

(22) Filed: Aug. 1, 2018

Related U.S. Application Data
(63) Continuation of application No. 15/557,308, filed on Sep. 11, 2017.
(60) Provisional application No. 62/132,042, filed on Mar. 12, 2015.

(51) Int. Cl. A61K 35/74; A61K 9/10; C12Q 1/689; C12N 1/20; G01N 33/50

(52) U.S. Cl. CPC A61K 35/74; G01N 2800/50; G01N 2800/06; A61K 9/10; G01N 2560/00; G01N 33/50; C12Q 1/689; G01N 2800/24; G01N 2333/195; C12N 1/20

(57) ABSTRACT

The present invention relates to bacterial compositions and methods of use thereof. The bacterial compositions may include two or more bacteria of the genera Faecalibacterium, Lachnospira, Veillonella or Rothia. The bacterial compositions may be used in treating gut dysbiosis, asthma, allergy, or atopy, or altering the gut microbiota, or populating the gastrointestinal tract, in a subject in need thereof.

### FIELD OF THE INVENTION

The present invention relates to bacterial compositions and methods of use thereof.

### BACKGROUND

Asthma is the most prevalent chronic disease among children and affects 235 million people worldwide¹. The striking difference in prevalence of asthma between developed and developing countries² highlights the influence of environmental factors, including diet and antibiotics use during infancy, which alters early microbial exposure and promotes development of immune hypersensitivities³. Recent studies in mice have implicated a ‘critical window’ early in life where the effects of gut microbial changes (dysbiosis) are most influential in immune development and experimental asthma⁴. Shifts in the gut microbiome and in gut microbe-derived compounds, including short-chain fatty acids (SCFA), have been implicated in a number of diseases⁵, including asthma⁶, although it is unclear whether these changes precede asthma and if they are involved in human asthma.

### SUMMARY

The present invention provides, in part, bacterial compositions and methods of use thereof. The bacterial compositions may be used, without limitation, to alter the gut microbiota, to populate the gastrointestinal tract, or to diagnose or, treat gut dysbiosis, asthma, allergy, or atopy in a subject in need thereof.

In one aspect, there is provided a method of treating one or more of gut dysbiosis, asthma, allergy, or atopy in a subject in need of such treatment, by administering to the subject an effective amount of a bacterial composition including two or more bacteria of the genera Faecalibacterium, Lachnospira, Veillonella or Rothia.

In an alternative aspect, there is provided a method of altering the gut microbiota in a subject in need of such treatment, by administering to the subject an effective amount of a bacterial composition including two or more bacteria of the genera Faecalibacterium, Lachnospira, Veillonella or Rothia.

In an alternative aspect, there is provided a method for populating the gastrointestinal tract of a subject in need of such treatment, by administering to the subject an effective amount of a bacterial composition including two or more bacteria of the genera Faecalibacterium, Lachnospira, Veillonella or Rothia.

In some embodiments, the subject is undergoing, will undergo, or has undergone antibiotic therapy.

In some embodiments, the subject is a human fetus, a human infant, or a pregnant female.

In some embodiments, the human infant is less than one year old.

In some embodiments, the bacterial composition is administered prophylactically.

In some embodiments, the bacterial composition is administered orally or rectally.

In some embodiments, the bacterial composition is formulated as a liquid suspension.

In some embodiments, the bacterial composition includes two or more of Faecalibacterium prausnitzii, Lachnospira multipara, Veillonella parvula, or Rothia mucilaginosa.

In some embodiments, the method includes administering to the subject an effective amount of a bacterial composition including three or more bacteria of the genera Faecalibacterium, Lachnospira, Veillonella or Rothia.

In some embodiments, the bacterial composition includes three or more of Faecalibacterium prausnitzii, Lachnospira multipara, Veillonella parvula, or Rothia mucilaginosa.

In some embodiments, the method includes administering to the subject an effective amount of a bacterial composition including bacteria of the genera Faecalibacterium, Lachnospira, Veillonella and Rothia.

In some embodiments, the bacterial composition includes Faecalibacterium prausnitzii, Lachnospira multipara, Veillonella parvula, and Rothia mucilaginosa.

In some embodiments, the administering results in an increase in the population of at least one or more of bacteria of the genera Faecalibacterium, Lachnospira, Veillonella or Rothia in the subject.

In some embodiments, the increase is determined using quantitative polymerase chain reaction.

In some embodiments, the increase is monitored by the detection of a metabolite present in a sample from said subject.

In some aspects, there is provided a bacterial composition including two or more bacteria of the genera Faecalibacterium, Lachnospira, Veillonella and Rothia, in combination with a carrier.

In some embodiments, the bacteria are present in an amount effective for treating gut dysbiosis, asthma, allergy, or atopy, or altering the gut microbiota, or populating the gastrointestinal tract, in a subject in need thereof.

In some embodiments, the bacterial composition is for use in treating gut dysbiosis, asthma, allergy, or atopy, or in altering the gut microbiota, or in populating the gastrointestinal tract, in a subject in need thereof.

In some embodiments, the bacteria are substantially pure.

In some aspects, there is provided a method of determining the likelihood of development of gut dysbiosis, asthma, allergy, or atopy in a subject, by determining the levels of two or more bacteria of the genera Faecalibacterium, Lachnospira, Veillonella or Rothia in a sample from said subject, and comparing said levels to a reference or a healthy subject, where a decrease in the levels of the bacteria indicates the likelihood of development of gut dysbiosis, asthma, allergy, or atopy.

In some embodiments, the method further includes determining the levels of a metabolite present in a sample from the subject.

In some embodiments, the method further includes administering an effective amount of the composition of claim 18 or 19 to a subject determined to have an increased likelihood of development of gut dysbiosis, asthma, allergy, or atopy.

This summary does not necessarily describe all features of the invention.

### BRIEF DESCRIPTION OF THE DRAWINGS

FIG. 1 is a schematic showing the classification of study participants, in which 319 selected subjects were classified into four clinical phenotypes based on skin prick tests and wheeze data at 1-year of age: Controls, Atopy+Wheeze (AW), Atopy only, and Wheeze only. The asthma predictive index (API) was calculated based on data at 3-years of age (Odds ratios compared to controls calculated the risk of each group to be diagnosed with asthma at school age (Odds ratios compared to controls: AW, 13.5 [p<0.001; 95% CI: 3.2 to 57.4]; Wheeze only, 2.7 (ns), Atopy only, 2.4 (ns)).

FIG. 2A is a multivariate analysis by PCA of the fecal microbiota across the four clinical phenotypes at 3-months. No differences were observed between the microbiotas of the four phenotypes.

FIG. 2B shows box plots of alpha diversity (Shannon Diversity Index) among the four clinical phenotypes at 3-months, where upper and lower “hinges” correspond to the first and third quartiles (the 25th and 75th percentiles). No differences in alpha diversity were observed.

FIG. 2C shows the relative abundances of bacterial families within the top 100 OTUs among the four phenotypes at 3-months. Statistical analysis between the four groups found significant differences between bacterial groups of low abundance in the control and atopic-wheezing groups (denoted in the zoomed-in sections of the bars).

FIG. 2D shows qPCR quantification of selected genera relative to total 16S amplification, in all AW fecal samples and a randomly selected subset of control fecal samples at 3-months (n_{CTRL}=20 n_{AW}=21) and 1-year (n_{CTRL}=19 n_{AW}=21) [Center values are presented as means±s.e.m (two-tailed Mann Whitney test; * p<0.05, ***p<0.001)].

FIG. 2E shows a heatmap of the top 30 most significant differentially abundant genes (KOs) obtained by PICRUSt analysis of the same subset of samples in C, at 3 months of age. Heatmap intensities represent variance-stabilized KO abundances. Hierarchical clustering of the subjects was based on Euclidean distance using the complete linkage method.

FIG. 2F shows a heatmap of the top 30 most significant differentially abundant genes (KOs) obtained by PICRUSt analysis of the same subset of samples in C, at 1 year of age. Heatmap intensities represent variance-stabilized KO abundances. Hierarchical clustering of the subjects was based on Euclidean distance using the complete linkage method. The capacity of this analysis to discriminate between atopic-wheezers and controls only occurs at 3 months but not at 1 year of age.

FIG. 3A shows the concentrations of the three most abundant SCFAs in feces of a subset of AW and control samples at 3-months of age (n_{AW}=13, n_{control}=13), measured by gas chromatography and normalized to feces weight.

FIG. 3B shows the relative concentrations of metabolites of known microbial origin or contribution detected in the urine of a subset of AW and control samples at 3-months of age (n_{AW}=19, n_{control}=16). Metabolomics data are shown as scaled intensities normalized to osmolality, measured by ultra-high performance liquid chromatography-tandem mass spectrometry. The superscript number next to each metabolite in the plot titles denotes the biochemical pathway the metabolite is involved in, as follows: 1) Secondary bile acid metabolism; 2) Hemoglobin metabolism; 3) Phenylalanine metabolism; 4) Histidine metabolism; 5) Food component. Shapiro-Wilk test for normality was performed. Statistics shown in all graphs are based on two-tailed Mann-Whitney Wilcoxon (if not normally distributed) or unpaired two-tailed t-test (if normally distributed); center values presented as means±s.e.m * p<0.05, ** p<0.01).

FIG. 4A shows bacterial family relative abundance in feces from mice (3 week-old pups) from parents previously inoculated with feces of an AW 3 month-old infant (AW), or with the same sample plus a live mixture of Lachnospira multipara, Veillonella parvula, Rothia mucilaginosa and Faecalibacterium prauznitzii (AW+FLVR). An evident change in family composition was observed between animals colonized with or without FLVR.

FIG. 4B shows that the percent abundance of Lachnospira sp., Veillonella sp., Rothia sp. and Faecalibacterium sp. was elevated in mouse pups born to parents inoculated with FLVR.

FIG. 4C shows cellular counts in the bronchoalveolar lavage (BAL) of mice harbouring the two different Microbial communities (AW or AW+FLVR) after a 3-week OVA immunization regime to induce airway inflammation. Naive mice received an immunization regime with saline.

FIG. 4D shows total cell differential counts in the BAL. Stars denote a significant decrease in lymphocytes (*) and neutrophils (****) between the AW and AW+FLVR groups (*p<0.05, ****p<0.0001).

FIG. 4E shows the total histopathological scores and representative haematoxylin and eosin-stained lung sections. Arrow shows the presence of significant inflammatory infiltrate in mice that were given the AW inoculum. The same inoculum with the addition of FLVR bacteria resulted in a reduced inflammatory infiltrate. Scale bar=300 μm.

FIG. 4F shows cytokine concentration in lung tissue homogenates measured by multiplexed cytometric bead array and normalized to total protein concentration.

FIG. 4G shows serum concentration of OVA-specific IgE, IgG1, and IgG2a measured by ELISA. C-G) Center values described as means±s.e.m from two independent experiments (n_{control}=8, n_{AW}=18, n_{AW+FLVR}=28). Samples represent biological replicates. Stars without a bracket denote significant difference in relation to naïve mice (ANOVA and Tukey multiple comparisons test, *p<0.05, **p<0.01, ***p<0.001, ****p<0.0001).

FIG. 5 shows gut microbial and host metabolic changes in the first year of life. A) Multivariate analysis by PCA of the 3-month and 1-year gut microbiota of 319 children, statistically compared by permanova (p<0.001). B) PCA of the gut microbial genetic composition of 319 children at 3-months and 1-year of age (p<0.01) C) PCA of the urinary metabolomic profile of 34 children at 3-months and 1-year of age (n_{3 mo}=35, n_{1 yr}=33); p<0.01).

FIG. 6A shows that alpha diversity, compared at 3-months and 1-year of age using the Shannon Diversity Index, statistically confirmed by two-tailed Mann Whitney Wilcoxon (p<0.05, shown as box plots, upper and lower “hinges” correspond to the first and third quartiles (the 25th and 75th percentiles).

FIG. 6B shows relative abundances of the top 100 OTUs represented by eight bacterial families and evidences the drastic shift in microbiota that occurs between 3 months and 1 year.

FIG. 6C shows a heatmap displaying the top 10 statistically significant (mt test; p<0.005) differentially abundant OTUs between 3-months and 1-year of age. Each rectangle is one subject.

FIG. 7A shows the PCA of the gut microbiota among the four clinical phenotypes at 1-year and the absence of overall microbiota shifts according to the differences in phenotypes.

FIG. 7B shows the alpha diversity (Shannon Diversity Index) among the four clinical phenotypes at 1-year (shown as box plots, upper and lower “hinges” correspond to the first and third quartiles (the 25th and 75th percentiles). No differences were observed among the four groups.

FIG. 7C shows the relative abundances of bacterial families (within the top 100 OTUs) among the four phenotypes at 1-year. Statistical analysis of the abundance of bacterial taxa did not yield significant differences between any of the groups.

FIG. 8 shows the relative abundance of bacterial genera within the top 100 OTUs among the four phenotypes at 3-months.

FIG. 9 shows the relative abundance of bacterial genera within the top 100 OTUs among the four phenotypes at 1 year.

FIG. 10 shows PICRUSt-predicted KEGG functional categories. PICRUSt-predicted KEGG functional categories with significant differences in relative abundance between controls and AWs (n_{CTRL}=20, n_{AW}=21; Welch t-test, q-value <0.05). Displayed are barplots of the categories with a difference in mean proportion >0.01% at 3-months.

FIG. 11A shows the nucleic acid sequence of Faecalibacterium praussnitzii ATCC 27766 16S ribosomal RNA, GenBank Accession Number X85022.1 (SEQ ID NO: 1).

FIG. 11B shows the nucleic acid sequence of Faecalibacterium praussnitzii ATCC 27768 16S ribosomal RNA, GenBank Accession Number AJ413954.1 (SEQ ID NO: 2).

FIG. 11C shows the nucleic acid sequence of Lachnospira multipara partial 16S ribosomal RNA, type strain DSM3073T, GenBank Accession Number FR733699.1 (SEQ ID NO: 3).

FIG. 11D shows the nucleic acid sequence of Veillonella parvula strain ATCC 10790 16S ribosomal RNA gene, NCBI Reference Sequence NR_043332.1 SEQ ID NO: 4).

FIG. 11E shows the nucleic acid sequence of Rothia mucilaginosa strain DSM 20746 16S ribosomal RNA gene, partial sequence NCBI Reference Sequence: NR_044873.1.

### DETAILED DESCRIPTION

The present invention provides, in part, bacterial compositions and methods of use thereof. The bacterial compositions may be used, without limitation, to alter the gut microbiota, to populate the gastrointestinal tract, or to diagnose or treat gut dysbiosis, asthma, allergy, or atopy in a subject in need thereof.

Bacterial Compositions and Uses Thereof

Bacterial compositions, as described herein, may include one or more bacteria of the family Ruminococcaceae, or one or more bacteria of the family Lachnospiraceae, or one or more bacteria of the family Veillonellaceae, or one or more bacteria of the family Micrococcaceae.

In some embodiments, bacterial compositions as described herein may include one or more bacteria of the genera Faecalibacterium, one or more bacteria of the genera Lachnospira, one or more bacteria of the genera Veillonella, or one or more bacteria of the genera Rothia.

In some embodiments, bacterial compositions as described herein may include two or more bacteria of the genera Faecalibacterium, Lachnospira, Veillonella or Rothia.

In some embodiments, bacterial compositions as described herein may include three or more bacteria of the genera Faecalibacterium, Lachnospira, Veillonella or Rothia.

In some embodiments, bacterial compositions as described herein may include four or more bacteria of the genera Faecalibacterium, Lachnospira, Veillonella and Rothia.

In some embodiments, bacterial compositions as described herein may include the following combinations of bacteria of the genera Faecalibacterium, Lachnospira, Veillonella or Rothia: Faecalibacterium and Lachnospira; Faecalibacterium and Veillonella; Faecalibacterium and Rothia; Lachnospira and Veillonella; Lachnospira and Rothia; Veillonella and Rothia; Faecalibacterium, Lachnospira and Veillonella; Faecalibacterium, Lachnospira and Rothia; Faecalibacterium, Veillonella and Rothia; Lachnospira, Veillonella and Rothia; Faecalibacterium, Lachnospira, Veillonella and Rothia.

In some embodiments, bacteria of the genera Faecalibacterium may include Faecalibacterium prausnitzii (previously also known as Fusobacterium prausnitzii, Bacillus mucosus anaerobius, or Bacteroides praussnitzii) or operational taxonomic unit (OTU) encompassing said species.

In some embodiments, the Faecalibacterium may include Faecalibacterium sp. CAG:1138 (also known as Faecalibacterium sp. MGS:1138), Faecalibacterium sp. CAG:82 (also known as Faecalibacterium sp. MGS:82), Faecalibacterium sp. CAG:74 (also known as Faecalibacterium sp. MGS:74), Faecalibacterium sp. DJF VR20, Faecalibacterium sp. canine oral taxon 147, or Faecalibacterium sp. MC 41.

In some embodiments, the Faecalibacterium prausnitzii may include the strains Faecalibacterium prausnitzii L2-6, Faecalibacterium cf. prausnitzii KLE1255, Faecalibacterium prausnitzii SL3/3, Faecalibacterium prausnitzii M21/2, or Faecalibacterium prausnitzii A2-165.

In some embodiments, the Faecalibacterium prausnitzii may be the Faecalibacterium prausnitzii deposited with the American Type Culture Collection (ATCC) under ATCC 27766 or ATCC 27768.

In some embodiments, the Faecalibacterium prausnitzii ATCC 27766 may include the 16S rRNA gene set forth under GenBank accession number X85022.1 (SEQ ID NO: 1).

In some embodiments, the Faecalibacterium prausnitzii may include a 16S rRNA gene having at least 95%, 96%, 97%, 98% or 99% sequence identity to the 16S rRNA gene set forth under GenBank accession number X85022.1 (SEQ ID NO: 1).

In some embodiments, the Faecalibacterium prausnitzii ATCC 27768 may include the 16S rRNA gene set forth under GenBank accession number AJ413954.1 (SEQ ID NO: 2).

In some embodiments, the Faecalibacterium prausnitzii may include a 16S rRNA gene having at least 95%, 96%, 97%, 98% or 99% sequence identity to the 16S rRNA gene set forth under GenBank accession number AJ413954.1 (SEQ ID NO: 2).

In some embodiments, bacteria of the genera Lachnospira may include Lachnospira multipara (previously known as Lachnospira multiparis) or Lachnospira pectinoschiza, or operational taxonomic unit (OTU) encompassing said species.

In some embodiments, the Lachnospira multipara may be Lachnospira multipara D32, Lachnospira multipara LB2003, Lachnospira multipara MC2003, or Lachnospira multipara DSM-3073.

In some embodiments, the Lachnospira multipara may be the Lachnospira multipara deposited with the ATCC under ATCC 19207 or DSM-3073

In some embodiments, the Lachnospira multipara DSM-3073 may include the 16S rRNA gene, type strain DSM3073T, set forth under GenBank accession number FR733699.1 (SEQ ID NO: 3).

In some embodiments, the Lachnospira multipara may include a 16S rRNA gene having at least 95%, 96%, 97%, 98% or 99% sequence identity to the 16S rRNA gene set forth under GenBank accession number FR733699.1 (SEQ ID NO: 3).

In some embodiments, the Lachnospira pectinoschiza may be the Lachnospira pectinoschiza, strain 150-1.

In some embodiments, the Lachnospira pectinoschiza may be the Lachnospira pectinoschiza deposited with the ATCC under ATCC 49827.

In some embodiments, bacteria of the genera Veillonella may include Veillonella parvula, Veillonella atypica, Veillonella parvula parvula, Veillonella di spar, Veillonella rogosae, Veillonella seminalis, Veillonella sp. oral taxon 780 str. F0422, Veillonella tobetsuensis, Veillonella montpellierensis, Veillonella magna, Veillonella sp. oral taxon 158 str. F0412, Veillonella ratti, Veillonella criceti, or operational taxonomic unit (OTU) encompassing said species.

In some embodiments, the Veillonella parvula may be Veillonella parvula ATCC 10790/DSM 2008/JCM 12972/Te3, Veillonella parvula ACS-068-V-Sch12, Veillonella parvula ATCC 17745, or Veillonella parvula HSIVP1.

In some embodiments, the Veillonella parvula may be the Veillonella parvula deposited with the ATCC under ATCC 10790.

In some embodiments, the Veillonella parvula ATCC 10790 may include the 16S rRNA gene set forth under GenBank accession number NR_043332.1 (SEQ ID NO: 4).

In some embodiments, the Veillonella parvula may include a 16S rRNA gene having at least 95%, 96%, 97%, 98% or 99% sequence identity to the 16S rRNA gene set forth under GenBank accession number NR_043332.1 (SEQ ID NO: 4).

In some embodiments, the Veillonella atypica may be Veillonella atypica KON/ATCC 17744/DSM 20739/NCTC 11830, Veillonella atypica D15, Veillonella atypica ACS-049-V-Sch6, or Veillonella atypica ACS-134-V-Col7a.

In some embodiments, the Veillonella dispar may be Veillonella dispar ATCC 17748, or Veillonella dispar DORA_11.

In some embodiments, the Veillonella rogosae may be Veillonella rogosae CCUG 54233/DSM 18960/JCM 15642/Veillonella sp. NVG 05cf.

In some embodiments, the Veillonella seminalis may be Veillonella seminalis ACS-216-V-Col6b (also known as Veillonella ratti ACS-216-V-Col6b), or Veillonella seminalis CIP 107810/MG 28162/Veillonella sp. ADV 4313.2/Veillonella sp. VA109.

In some embodiments, the Veillonella tobetsuensis may be Veillonella tobetsuensis ATCC BAA-2400/JCM 17976/Veillonella sp. A16/Veillonella sp. B4/Veillonella sp. IM-2011/Veillonella sp. JCM 17976/Veillonella sp. Y6/strain B16.

In some embodiments, the Veillonella montpellierensis may be Veillonella montpellierensis CCUG 48299/CIP 107992/DSM 17217/Veillonella sp. 2001-112662/Veillonella sp. ADV 2216.03/Veillonella sp. ADV 281.99/Veillonella sp. ADV 3198.03/strain ADV 281.99, or Veillonella montpellierensis DNF00314.

In some embodiments, the Veillonella magna may be Veillonella magna DSM 19857 (also known as Veillonella magna lac18).

In some embodiments, the Veillonella ratti may be Veillonella ratti ATCC 17746/DSM 20736/JCM 6512/NCTC 12019.

In some embodiments, the Veillonella criceti may be Veillonella criceti ATCC 17747/DSM 20734/JCM 6511/NCTC 12020.

In some embodiments, bacteria of the genera Rothia may include Rothia mucilaginosa (previously known as Stomatococcus mucilaginosus or Micrococcus mucilaginous), Rothia dentocariosa, Rothia aeria, Rothia nasimurium, Rothia marina, Rothia terrae, Rothia endophytica, Rothia amarae, Rothia arfidiae, Rothia sp. CCUG 25688, Rothia sp. ChDC B201, Rothia sp. oral clone BP1-65, or operational taxonomic unit (OTU) encompassing said species.

In some embodiments, the Rothia mucilaginosa may be Rothia mucilaginosa (strain DY-18), Rothia mucilaginosa ATCC 25296/CCM 2417/CCUG 20962/CIP 71.14, Rothia mucilaginosa M508, Rothia mucilaginosa CC87LB, Rothia mucilaginosa M1710.

In some embodiments, the Rothia mucilaginosa may be the Rothia mucilaginosa deposited with the ATCC under ATCC 49040 or ATCC 25296.

In some embodiments, the Rothia mucilaginosa ATCC 49040 may include the 16S rRNA gene, type strain DSM 20746, set forth under GenBank accession number NR_044873.1 (SEQ ID NO: 5).

In some embodiments, the Rothia mucilaginosa ATCC 49040 may include a 16S rRNA gene having at least 95%, 96%, 97%, 98% or 99% sequence identity to the 16S rRNA gene set forth under GenBank accession number NR_044873.1 (SEQ ID NO: 5).

In some embodiments, the Rothia dentocariosa may be Rothia dentocariosa (strain ATCC 17931/CDC X599/XDIA), Rothia dentocariosa M567.

In some embodiments, the Rothia aeria may be Rothia aeria F0474, Rothia aeria F0184 (also known as Rothia sp. oral taxon 188 str. F0184), Rothia aeria DSM 14556/GTC 867/JCM 11412/Rothia aerius/strain A1-17B.

In some embodiments, the Rothia nasimurium may be Rothia nasimurium CCUG 35957/CIP 106912/JCM 10909/Rothia sp. M7SW7a/Rothia-like sp. CCUG 35957.

In some embodiments, the Rothia marina may be Rothia marina DSM 21080/KCTC 19432/Rothia sp. G7/Rothia sp. JSM 078151/strain JSM 078151.

In some embodiments, the Rothia terrae may be Rothia terrae BCRC 17588/JCM 15158/LMG 237081 Rothia sp. L-143/strain L-143.

In some embodiments, the Rothia endophytica may be Rothia endophytica DSM 26247/JCM 18541/Rothia sp. YIM 67072/YIM 67072.

In some embodiments, the Rothia amarae may be Rothia amarae AS 4.1721/CCUG 47294/JCM 11375/Kocuria sp. j-18/strain J18.

In some embodiments, bacterial compositions as described herein may include the following combinations of Faecalibacterium prausnitzii, Lachnospira multipara, Veillonella parvula, or Rothia mucilaginosa: Faecalibacterium prausnitzii and Lachnospira multipara; Faecalibacterium prausnitzii; and Veillonella parvula; Faecalibacterium prausnitzii and Rothia mucilaginosa; Lachnospira multipara and Veillonella parvula; Lachnospira multipara and Rothia mucilaginosa; Veillonella parvula and Rothia mucilaginosa; Faecalibacterium prausnitzii, Lachnospira multipara and Veillonella parvula; Faecalibacterium prausnitzii, Lachnospira and Rothia mucilaginosa; Faecalibacterium prausnitzii, Veillonella parvula and Rothia mucilaginosa; Lachnospira multipara, Veillonella parvula and Rothia mucilaginosa; Faecalibacterium prausnitzii, Lachnospira multipara, Veillonella parvula and Rothia mucilaginosa.

By “operational taxonomic unit” or “OTU” is meant classification of microbes within the same, or different, OTUs using techniques, as described herein, or known in the art. OTU refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S rRNA sequence of a bacterium, or a portion of the 16S rRNA sequence. In other embodiments, the entire genomes of two organisms can be sequenced and compared. In another embodiment, select regions such as multilocus sequence tags (MLST), specific genes, or sets of genes may be genetically compared. In 16S rRNA embodiments, OTUs that share >97% average nucleotide identity across the entire 16S rRNA or some variable region of the 16S rRNA are considered the same OTU³¹⁻³². In embodiments involving the complete genome, MLSTs, specific genes, or sets of genes OTUs that share >95% average nucleotide identity are considered the same OTU³²⁻³³. OTUs are in some cases defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence. “16S sequencing” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi. In some. embodiments, OTUs may be determined using CrunchClust²⁹ and classified against the Greengenes Database³⁰ according to 97% similarity.

In some embodiments, a bacterial composition as described herein may include bacteria comprising 16S rRNA gene sequences substantially identical to the sequences set forth in one or more of SEQ ID NOs. 1 to 5. By “substantially identical” is meant a nucleic acid sequence that differs from a reference sequence only by one or more conservative substitutions, as discussed herein, or by one or more non-conservative substitutions, deletions, or insertions located at positions of the sequence that do not destroy the biological function of the nucleic acid molecule. Such a sequence can be any integer at least 70%, 75%, 80%, 85%, 90% or over 95%, or more generally at least 95%, 96%, 97%, 98%, 99%, or 100% identical when optimally aligned at the nucleotide level to the sequence used for comparison using, for example, FASTA. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al.³⁴.

In some embodiments, a bacterial composition as described herein may include bacteria as described herein, present in treated fecal material from a healthy donor or individual. Such bacterial compositions may be “directly isolated” and not resulting from any culturing or other process that results in or is intended to result in replication of the population after obtaining the fecal material. In some embodiments, bacteria as described herein include bacterial spores.

In some embodiments, a bacterial composition as described herein may include human bacterial strains. In alternative embodiments, a bacterial composition as described herein may include bacterial strains not generally found in humans.

In some embodiments, a bacterial composition as described herein may include bacteria capable of colonizing the gut of a subject receiving the bacterial composition.

In some embodiments, a bacterial composition as described herein may include live bacteria.

In some embodiments, a bacterial composition as described herein may include substantially pure bacteria of the genera Faecalibacterium, Lachnospira, Veillonella and/or Rothia. By “substantially pure” or “isolated” is meant bacteria of the genera Faecalibacterium, Lachnospira, Veillonella and/or Rothia that are separated from the components that naturally accompany it, in for example, fecal matter or in the gut. Typically, a bacterial composition as described herein is substantially pure when it is at least 50%, 60%, 70%, 75%, 80%, or 85%, or over 90%, 95%, or 99% by weight, of the total material in a sample. A substantially pure bacterial composition, as described herein, can be obtained, for example, by extraction from a natural source, such as fecal material from a healthy individual, or from bacterial cultures, for example, cultures of any of the bacteria described herein, such as Faecalibacterium prausnitzii, Lachnospira multipara, Veillonella parvula, and/or Rothia mucilaginosa.

Bacterial compositions, as described herein, may be used to alter the gut microbiota, to populate the gastrointestinal tract, or to diagnose or treat gut dysbiosis, asthma, allergy, or atopy in a subject in need thereof In some embodiments, treating gut dysbiosis may result in the prevention of asthma, allergy or atopy in the subject.

The term asthma refers to a common chronic inflammatory disease of the airways characterized by variable and recurring symptoms, reversible airflow obstruction and bronchospasm. Common symptoms include wheezing, coughing, chest tightness, and shortness of breath.

The term dysbiosis refers to microbial imbalance on or inside the body and most commonly refers to a condition in the digestive tract or gut. Any disruption from a healthy (e.g., ideal) state of the microbiota or microbiome can be considered a dysbiosis, even if such dysbiosis does not result in a detectable decrease in health. A state of dysbiosis may be unhealthy, it may be unhealthy under only certain conditions, or it may prevent a subject from becoming healthier. Dysbiosis may be due to, for example, a decrease in diversity. It has been associated with illnesses, such as inflammatory bowel disease' colitis, chronic fatigue syndrome, obesity, cancer and asthma.

The term atopy refers to a genetic predisposition to a hypersensitivity response to allergens, such as environmental allergens. Atopy includes, but is not limited to atopic dermatitis (eczema), allergic rhinitis (hay fever), allergic asthma and diseases associated with atopy, such as food allergies, allergic conjunctivitis and eosinophilic esophagitis.

The term allergy refers to an abnormal immune reaction to an allergen.

By “populating the gastrointestinal tract” is meant establishing a healthy state of the microbiota or microbiome in a subject. In some embodiments, populating the gastrointestinal tract includes increasing or decreasing the levels of specific bacteria in the gastrointestinal tract of a subject. In some embodiments, populating the gastrointestinal tract includes increasing the levels of the bacteria described herein in the gastrointestinal tract of a subject.

By “altering the gut microbiota” is meant any change, either increase or decrease, of the microbiota or microbiome in a subject. In some embodiments, altering the gut microbiota includes increasing or decreasing the levels of specific bacteria, such as in the gastrointestinal tract of a subject. In some embodiments, altering the gut microbiota includes increasing the levels of the bacteria described herein in the gastrointestinal tract of a subject.

By “increase,” “increasing”, “decrease” or “decreasing” is meant a change in the levels of specific bacteria in the gastrointestinal tract of a subject. An increase or decrease may include a change of any value between 10% and 100%, or of any value between 30% and 60%, or over 100%, for example, a change of about 10%, 20% 30%, 40%, 50%, 60%, 70%, 80%, 90%, 95% or more, when compared to a control. In some embodiments, the increase or decrease may be a change of about 1-fold, 2-fold, 5-fold, 10-fold, 100-fold, or more, when compared to a control.

“Microbiota” refers to the community of microorganisms that occur (sustainably or transiently) in and on an animal subject, typically a mammal such as a human, including eukaryotes, archaea, bacteria, and viruses (including bacterial viruses, such as phage). “Microbiome” refers to the genetic content of the communities of microbes that live in and on the human body, both sustainably and transiently, including eukaryotes, archaea, bacteria, and viruses (including bacterial viruses, such as phage), where “genetic content” includes genomic DNA, RNA such as ribosomal RNA, the epigenome, plasmids, and other types of genetic information.

The terms “treatment,” “treating” or “therapy” encompass prophylactic, palliative, therapeutic, and nutritional modalities of administration of the bacterial compositions described herein. Accordingly, treatment includes amelioration, alleviation, reversal, or complete elimination of one or more of the symptoms in a subject diagnosed with, or known to have, gut dysbiosis, asthma, allergy, or atopy, or be considered to derive benefit from the alteration of gut microbiota. In some embodiments, treatment includes reduction of one or more symptoms of gut dysbiosis, asthma, allergy, or atopy by 10%, 20% 30%, 40%, 50%, 60%, 70%, 80%, 90%, 95% or more. Treatment also includes prevention or delay of the onset of one or more symptoms of gut dysbiosis, asthma, allergy, or atopy.

As used herein, a subject may be a mammal, such as a human, non-human primate (e.g., monkey, baboon, or chimpanzee), rat, mouse, rabbit, guinea pig, gerbil, hamster, cow, horse, pig, sheep, goat, dog, cat, etc. In some embodiments, the subject is a patient. The subject may be an infant, such as a human infant less than one year old, or less than three months old. In some embodiments, the subject may be a human infant at any age from 1 day to 350 days old, such as 1 day, 10 days, 20 days, 30 days, 40 days, 50 days, 60 days, 70 days, 80 days, 90 days, 100 days, 110 days, 120 days, 130 days, 140 days, 150 days, 160 days, 170 days, 180 days, 190 days, 200 days, 210 days, 220 days, 230 days, 240 days, 250 days, 260 days, 270 days, 280 days, 290 days, 300 days, 310 days, 320 days, 330 days, 340 days, or 350 days old. In some embodiments, the subject may be a fetus. In some embodiments, the subject may be a female, such as a pregnant female. In some embodiments, the subject may be a pregnant female with a family history of asthma, atopy, allergy or gut dysbiosis. In some embodiments, the subject may have undergone, be undergoing, or about to undergo, antibiotic therapy. The subject may be a clinical patient, a clinical trial volunteer, an experimental animal, etc. The subject may be suspected of having or at risk for gut dysbiosis, asthma, allergy, or atopy; be diagnosed with gut dysbiosis, asthma, allergy, or atopy; or be a control subject that is confirmed to not have gut dysbiosis, asthma, allergy, or atopy. Diagnostic methods for gut dysbiosis, asthma, allergy, or atopy, and the clinical delineation of such diagnoses are known to those of ordinary skill in the art. In some embodiments, the subject may be an individual considered to be benefitted by the alteration of gut microbiota. In some embodiments, the subject may be an individual considered to be benefitted by population of the gastrointestinal tract.

Pharmaceutical & Nutritional Compositions, Dosages & Administration

Bacterial compositions, as described herein, can be provided alone or in combination with other compounds or compositions, in the presence of a carrier, in a form suitable for administration to a subject, as described herein. Where the subject is a fetus, a bacterial composition as described herein may be administered to the mother (i.e., the subject may be a pregnant female).

In some embodiments, a bacterial composition, as described herein, may be a therapeutic, prophylactic, nutritional or probiotic composition.

In some embodiments, a bacterial composition may be a therapeutic, prophylactic, nutritional or probiotic composition including the bacteria of the genera Faecalibacterium, Lachnospira, Veillonella and/or Rothia.

In some embodiments, a bacterial composition may be a therapeutic, prophylactic, nutritional or probiotic composition including Faecalibacterium prausnitzii, Lachnospira multipara, Veillonella parvula, and/or Rothia mucilaginosa.

If desired, a bacterial composition as described herein may be combined with more traditional and existing therapies for gut dysbiosis, asthma, allergy, or atopy.

In some embodiments, a bacterial composition as described herein may be combined with one or more therapies for asthma, including without limitation, short-acting bronchodilators, beta2-agonists, inhaled steroids, long-acting bronchodilators, anti-leukotrienes, anti-IgE therapy, oral corticosteroids, or theophyllines. In some embodiments, a bacterial composition as described herein may be combined with one or more of Fenoterol, Formoterol, Ipratropium, Isoproterenol, Orciprenaline, Salbutamol, Salbutamol, Terbutaline, Budesonide, Fluticasone, Ciclesonide, Beclomethasone Dipropionate, Salmeterol, Montelukast, Zafirlukast, omalizumab, Prednisolone, or Prednisone.

In some embodiments, a bacterial composition as described herein may be combined with one or more therapies for allergy, including without limitation, antihistamines, decongestants, steroids, bronchodilators, mast cell stabilizers, or leukotriene modifiers. In some embodiments, a bacterial composition as described herein may be combined with one or more of cetirizine, ciclesonide, ketotifen, levocetirizine, fluticasone, furoate, epinephrine, clemastine, montelukast, budesonide, olopatadine, carbinoxamine maleate, mometasone, flunisolide, cromolyn sodium, triamcinolone, oxymetazoline, epinastine, dexamethasone, loratidine, desloratidine, diphenhydramine, beclomethasone, azelastine, loteprednol etabonate, fexofenadine, or neodrocromil sodium.

In some embodiments, a bacterial composition as described herein may be administered to a subject prior to, during, or subsequent to treatment with an antibiotic. In some embodiments, a bacterial composition as described herein may be combined with one or more antibiotic including, without limitation, streptomycin, ampicillin, amoxicillin, imipenem, piperacillin/tazobactam, ciprofloxacin, tetracyclines, chloramphenicol or ticarcillin.

The term probiotic herein is intended to mean one or more, or a mixture of, microorganisms that provide health benefits when consumed.

The bacterial compositions can be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical or nutraceutical practice may be employed to provide suitable formulations or compositions to administer a bacterial composition, as described herein, to subjects suffering from or presymptomatic for gut dysbiosis, asthma, allergy, or atopy. Any appropriate route of administration may be employed, for example, dermal, intranasal, inhalation aerosol, topical, gavage, rectal or oral administration.

The bacterial compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and application. Formulations may be in the form of liquid solutions or suspensions; for oral administration, formulations may be in the form of tablets or capsules; for pediatric oral administration, formulations may be in the form of liquids or suspensions; or for intranasal formulations, in the form of powders, nasal drops, or aerosols. The formulation may be a slow release formulation. In some embodiments, bacterial as described herein, can be formulated as pediatric formulations, such as liquid suspensions.

Bacterial compositions, as described herein, can be formulated as a nutraceutical composition, such as medical foods, nutritional or dietary supplements, food products or beverage products, and include a nutraceutically acceptable carrier. As used herein, a “nutraceutically acceptable carrier” refers to, and includes, any and all solvents, dispersion media, coatings, antibacterial and antifungal agents, isotonic and absorption delaying agents, and the like that are physiologically compatible. The compositions can include a nutraceutically acceptable salt, e.g., an acid addition salt or a base addition salt. In some embodiments, the nutraceutically acceptable carrier is suitable for pediatric use.

Bacterial compositions, as described herein, can be formulated as a pharmaceutical composition and include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt. In some embodiments, the pharmaceutically acceptable carrier is suitable for pediatric use.

Methods well known in the art for making formulations are found in, for example, Gennaro³⁵. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the compounds are administered to a subject in an amount sufficient to stop or slow gut dysbiosis, asthma, allergy, or atopy, depending on the disorder

An “effective amount” of a bacterial composition according to the invention includes an amount sufficient to colonize the gut of a subject for a suitable period of time as determined, for example, by detecting the presence of one or more bacteria of the genera Faecalibacterium, Lachnospira, Veillonella and/or Rothia in a sample, such as a fecal sample, from the subject at specific periods after administration.

In some embodiments, an effective amount includes a therapeutically effective amount or a prophylactically effective amount. A “therapeutically effective amount” refers to an amount effective, at dosages and for periods of time necessary, to achieve the desired therapeutic result, such as treatment, prevention, or amelioration of gut dysbiosis, asthma, allergy, or atopy. A therapeutically effective amount of a bacterial composition may vary according to factors such as the disease state, age, sex, and weight of the subject, and the ability of the bacterial composition to elicit a desired response in the individual. Dosage regimens may be adjusted to provide the optimum therapeutic response. A therapeutically effective amount is also one in which any toxic or detrimental effects of the bacterial composition are outweighed by the therapeutically beneficial effects.

A “prophylactically effective amount” refers to an amount effective, at dosages and for periods of time necessary, to achieve the desired prophylactic result, such as treatment, prevention, or amelioration of gut dysbiosis, asthma, allergy, or atopy. Typically, a prophylactic dose is used in subjects prior to or at an earlier stage of disease, so that a prophylactically effective amount may be less than a therapeutically effective amount.

A “probiotic” amount refers to an amount effective, at dosages and for periods of time necessary, to achieve the desired result, such as population of the gastrointestinal tract of a subject after, for example, antibiotic treatment, to normal levels. Typically, probiotic doses are administered at larges excess and may be significantly higher than prophylactically effective or therapeutically effective amounts.

A suitable range for therapeutically or prophylactically effective amounts, or probiotic amounts, of a bacterial composition, as described herein, may include without limitation at least about 10⁶, 10⁷, 10⁸, 10⁹, 10¹⁰, 10¹¹, 10¹², 10¹³, or 10¹⁴ colony forming units (cfus) of the bacteria, per unit dosage.

In some embodiments, dosages for live bacteria, in vegetative or spore forms, can be about lug to about 1000 mg, such as about 0.5 mg to about 5 mg, about 1 mg to about 1000 mg, about 2 mg to about 200 mg, about 2 mg to about 100 mg, about 2 mg to about 50 mg, about 4 mg to about 25 mg, about 5 mg to about 20 mg, about 10 mg to about 15 mg, about 50 mg to about 200 mg, about 200 mg to about 1000 mg, or about 1, 2, 3, 4, 5 or more than 5 g per dose or composition; or 0.001 mg to 1 mg, 0.5 mg to 5 mg, 1 mg to 1000 mg, 2 mg to 200 mg, or 2 mg to 100 mg, or 2 mg to 50 mg, or 4 mg to 25 mg, or 5 mg to 20 mg, or 10 mg to 15 mg, or 50 mg to 200 mg, or 200 mg to 1000 mg, or 1, 2, 3, 4, 5 or more than 5 g per dose or composition.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Accordingly, in some embodiments, suitable dosages include pediatric dosages or dosages suitable for administration to pregnant females. In some embodiments, suitable dosages include probiotic dosages, such as pediatric probiotic dosages. Dosage regimens may be adjusted to provide the optimum desired response. For example, a single bolus may be administered, several divided doses may be administered over time or the dose may be proportionally reduced or increased as indicated by the exigencies of the situation. It may be advantageous to formulate parenteral compositions in dosage unit form for ease of administration and uniformity of dosage.

The bacterial compositions may be administered daily or more frequently, such as twice or more daily.

The bacterial compositions may be administered prior to, during or after consumption of a food or beverage.

Detection Methods

Also provided herein are methods of determining the likelihood of development of gut dysbiosis, asthma, allergy, or atopy in a subject, by determining the levels of one or more bacteria of the genera Faecalibacterium, Lachnospira, Veillonella or Rothia in the subject, and comparing the determined levels to a reference or a healthy individual, such as an individual not diagnosed with gut dysbiosis, asthma, allergy, or atopy, where a reduction or decrease in the levels of one or more bacteria of the genera Faecalibacterium, Lachnospira, Veillonella or Rothia indicates an increased likelihood of development of gut dysbiosis, asthma, allergy, or atopy. In general, a statistically significant difference between the subject and the reference or healthy individual indicates that the subject is likely to develop gut dysbiosis, asthma, allergy, or atopy. In some embodiments, a difference of 1 or 2, on the logarithmic scale, between the subject and the reference or healthy individual may indicate a likelihood of development of gut dysbiosis, asthma, allergy, or atopy in a subject.

In some embodiments, the levels of two or more bacteria of the genera Faecalibacterium, Lachnospira, Veillonella or Rothia in a sample from a subject may be determined. In some embodiments, the levels of three or more bacteria of the genera Faecalibacterium, Lachnospira, Veillonella or Rothia in a sample from a subject may be determined. In some embodiments, the levels of bacteria of the genera Faecalibacterium, Lachnospira, Veillonella and Rothia in a sample from a subject may be determined.

In some embodiments, determining the likelihood of development of gut dysbiosis, asthma, allergy, or atopy in a subject, include determining the levels of one of more of a metabolite, such as fecal acetate, urinary urobilinogen or bile acids, such as deconjugated bile acids, where a reduced level of, or decrease in, fecal acetate, or an increase in urinary urobilinogen or bile acids, such as deconjugated bile acids indicates that the subject is likely to develop gut dysbiosis, asthma, allergy, or atopy.

By “determining” or “detecting” it is intended to include determining the presence or absence of a substance or quantifying the amount of a substance, such as one or more of the bacteria described herein, or a metabolite as described herein. The term thus refers to the use of the materials, compositions, and methods described herein or known in the art for qualitative and quantitative determinations. An increase or decrease may include a change of any value between 10% and 100%, or of any value between 30% and 60%, or over 100%, for example, a change of about 10%, 20% 30%, 40%, 50%, 60%, 70%, 80%, 90%, 95% or more, when compared to a control. In some embodiments, the increase or decrease may be a change of about 1-fold, 2-fold, 5-fold, 10-fold, 100-fold, or more, when compared to a control.

A subject determined to be likely to develop gut dysbiosis, asthma, allergy, or atopy may be treated with a bacterial composition, as described herein.

In some embodiments, the efficacy of the treatment may be monitored by determining the levels of one or more bacteria of the genera Faecalibacterium, Lachnospira, Veillonella or Rothia, or a metabolite, in a sample from the subject, and comparing the determined levels to previous determinations from the subject.

A “sample” can be any organ, tissue, cell, or cell extract isolated from a subject, such as a sample isolated from a mammal having, suspected of having, or having a predisposition to gut dysbiosis, asthma, allergy, or atopy. For example, a sample can include, without limitation, blood, urine, stool, saliva, or any other specimen, or any extract thereof, obtained from a patient (human or animal), test subject, or experimental animal. A “control” includes a sample obtained for use in determining base-line expression or activity. Accordingly, a control sample may be obtained from a healthy individual, such as an individual not diagnosed with gut dysbiosis, asthma, allergy, or atopy. A control also includes a previously established standard or reference. Accordingly, any test or assay may be compared with the established standard and it may not be necessary to obtain a control sample for comparison each time. The sample may be analyzed to detect the presence or levels of a Faecalibacterium, Lachnospira, Veillonella or Rothia gene, genome, polypeptide, nucleic acid molecule, such as a Faecalibacterium, Lachnospira, Veillonella or Rothia 16S rRNA molecule, using methods that are known in the art, such as quantitative PCR. The sample may be analyzed to detect the presence or levels of a metabolite, such as fecal acetate, urinary urobilinogen or bile acids, such as deconjugated bile acids.

The present invention will be further illustrated in the following examples.

### EXAMPLES

Materials and Methods

Methods

Study Design, Skin Prick Testing and Sample Selection:

The CHILD study is a multi-centre longitudinal, prospective, general population birth cohort study following infants from pregnancy to age 5-years with a total of 3,624 pregnant mothers recruited at 4 sites across Canada (Vancouver, Edmonton, Manitoba, Toronto). Detailed characteristics of the CHILD study have been previously described¹. Briefly, questionnaires were administered at recruitment, 36-weeks gestation, at 3, 6, 12, 18, 24, 30 months, and at 3, 4, and 5-years. In this way, data were obtained related to environmental exposures, psychosocial stresses, nutrition, and general health. In addition, at ages 1, 3, and 5-years, questionnaires validated in the International Study of Asthma and Allergies in Childhood (ISAAC)² were completed by the parent. At age 1, 3, and 5-years, each child was examined for evidence of atopic dermatitis, rhinitis or asthma. Trained staff performed skin testing for standardized inhalant allergens and common food allergens at 1, 3, and 5-years. 5-year data was not included in this study as it was not available for this entire cohort at the date of submission. The University of British Columbia/Children's and Women's Health Centre of British Columbia Research Ethics Board approved the research protocols for studies on human samples and each participating parent gave signed informed consent.

Inclusion/Exclusion Criteria:

Skin prick test results: At 1-year of age, children enrolled in the CHILD study were tested with 10 allergens (Alternaria tenuis, cat hair, dog epithelium, Dermatophagoides pteronyssinus, Dermatophagoides farinae, German cockroach, peanut, soybean, egg white, and cow's milk). A child was classified as “atopic” if he/she produced a wheat >2 mm for any of the ten allergens tested. Histamine was used as a positive control and glycerin as a negative control. Subjects that tested negative to histamine were not included in this cohort unless they tested positive (with a wheal >2 mm) for one of the 10 allergens listed above. If a subject tested positive to glycerin, the wheal size for glycerin was subtracted from the wheal size of any positive allergen response.

Wheeze questionnaires: If a child had wheezed with or without a cold during the first year of life (recorded via questionnaires answer by parents at 3, 6, and 12-months), the child was included in the wheezing group. Children were also included in the wheezing group if the CHILD clinician recorded a wheeze during the 1-year clinical assessment.

Biological samples: Both a 3-month and a 1-year stool sample were required for each child to be included in this cohort. For the control group, subjects from whom additional samples were collected by the CHILD study (such blood samples) were selected over subjects missing any of these samples.

Of the 3542 children meeting the eligibility criteria at birth, 1427 children had completed the CHILD study 1-year clinical assessment at the time of selection. 163 subjects were excluded due to incomplete skin prick test data or a positive response to glycerin or a negative response to histamine and all other allergens. The remaining 1264 subjects were grouped into the four clinical phenotypes, atopy+wheeze (AW) (n=35), atopy only (n=150), wheeze only (n=216), and controls (n=863) and assessed for the availability of a 3-month and a 1-year stool sample [N numbers for children with 3-month and 1-year stool samples available AW (n=25), atopy only (n=112), wheeze only (n=179), and controls (n=106)]. Subjects were then excluded from the study if, after preparation and sequencing of the 16S DNA, the sequence results were inadequate (i.e. not enough sequence reads per stool sample) [final N numbers, AW (n=22), atopy only (n=87), wheeze only (n=136), and controls (n=74)].

The subsets of samples for qPCR, SCFA, urine metabolomics, and PICRUSt analysis included all available AW samples and 20 or less randomly selected control samples. The number of samples selected depended upon the availability of the fecal or urine samples which tended to be very limited in this study of human infants.

Asthma Predictive Index (API):

Subjects in our sample cohort (n=319) were also classified according to the Asthma Predictive Index². A positive stringent API is defined by the following criteria: recurrent wheeze between the ages of 2 and 3 years, together with 1 of 2 major criteria or 2 of 3 minor criteria. Additionally, if a child was diagnosed with asthma at the 3-year clinical assessment they were also included in the positive API group whether or not they met the API criteria.

Recurrent wheezing: Recurrent wheezing is defined as ≥3 episodes of wheezing between the ages of 2 and 3. Questionnaires at 24 and 30-months, and 3-years of age were used to quantify the number of wheeze episodes between 2 and 3-years.

Major Criteria: Parental history of asthma (from either parent) or M.D. diagnosed childhood atopic dermatitis between the ages of 2 and 3.

Minor Criteria: ≥4% Eosinophilia, any episodes of wheezing apart from colds after 2-years, and M.D. diagnosed allergic rhinitis at 3-years of age.

16S Microbial Community Analysis

DNA was extracted from ˜50 mg of stool or ˜50 mg of stool-coated swab was cut from the total swab. Samples were mechanically lysed using the Mo-bio dry bead tubes (Mo Bio Laboratories, Carlsbad, Calif.) and the Fastprep homogenizer (FastPrep Instrument, MP Biochemicals, Solon, Ohio), prior to DNA extraction with the QIAGEN DNA Stool Mini Kit.

All samples were amplified by polymerase chain reaction (PCR) in triplicate using barcoded primer pairs flanking the V3 region of the 16S gene (Table 7) as previously described⁴.

TABLE 7
16S V3 region amplification primers and barcodes

| Forward | | Reverse | |
|---|---|---|---|
| Primer | | Primer | |
| ID | Barcoded primer | ID | Barcoded primer |
| 341F/A | CTGATCNNNNCCTACGGGAGGCAG | 518R/a | aaccccATTACCGCGGCTGCTG |
| | CAG (SEQ ID NO: 18) | | G (SEQ ID NO: 19) |
| 341F/B | AGCATCNNNNCCTACGGGAGGCAG | 518R/b | ccaacaATTACCGCGGCTGCTG |
| | CAG (SEQ ID NO: 20) | | G (SEQ ID NO: 21) |
| 341F/C | CGATTANNNNCCTACGGGAGGCAG | 518R/c | agttccATTACCGCGGCTGCTG |
| | CAG (SEQ ID NO: 22) | | G (SEQ ID NO: 23) |
| 341F/D | CATTCANNNNCCTACGGGAGGCAG | 518R/d | aceggcATTACCGCGGCTGCTG |
| | CAG (SEQ ID NO: 24) | | G (SEQ ID NO: 25) |
| 341F/E | AAGCTANNNNCCTACGGGAGGCAG | 518R/e | caactaATTACCGCGGCTGCTG |
| | CAG (SEQ ID NO: 26) | | G (SEQ ID NO: 27) |
| 341F/F | GCTGTANNNNCCTACGGGAGGCAG | 518R/f | ccacgcATTACCGCGGCTGCTG |
| | CAG (SEQ ID NO: 28) | | G (SEQ ID NO: 29) |
| 341F/G | ATGGCANNNNCCTACGGGAGGCAG | 518R/g | ctatacATTACCGCGGCTGCTG |
| | CAG (SEQ ID NO: 30) | | G (SEQ ID NO: 31) |
| 341F/H | GCCTAANNNNCCTACGGGAGGCAG | 518R/h | tacagcATTACCGCGGCTGCTG |
| | CAG (SEQ ID NO: 32) | | G (SEQ ID NO: 33) |
| 341F/I | GTAGCCNNNNCCTACGGGAGGCAG | 518R/i | atgtcaATTACCGCGGCTGCTG |
| | CAG (SEQ ID NO: 34) | | G (SEQ ID NO: 35) |
| 341F/J | AAGTGCNNNNCCTACGGGAGGCAG | 518R/j | ttaggcATTACCGCGGCTGCTG |
| | CAG (SEQ ID NO: 36) | | G (SEQ ID NO: 37) |
| 341F/K | ATTATANNNNCCTACGGGAGGCAG | 518R/k | ggctacATTACCGCGGCTGCTG |
| | CAG (SEQ ID NO: 38) | | G (SEQ ID NO: 39) |
| 341F/L | CCAGCANNNNCCTACGGGAGGCAG | 518R/l | acgataATTACCGCGGCTGCTG |
| | CAG (SEQ ID NO: 40) | | G (SEQ ID NO: 41) |
| 341F/M | TGGTCANNNNCCTACGGGAGGCAG | 518R/m | ctcagaATTACCGCGGCTGCTG |
| | CAG (SEQ ID NO: 42) | | G (SEQ ID NO: 43) |
| 341F/N | CCACTCNNNNCCTACGGGAGGCAG | 518R/n | ccgtccATTACCGCGGCTGCTG |
| | CAG (SEQ ID NO: 44) | | G (SEQ ID NO: 45) |
| 341F/O | CGCGGCNNNNCCTACGGGAGGCAG | 518R/o | tgaccaATTACCGCGGCTGCTG |
| | CAG (SEQ ID NO: 46) | | G (SEQ ID NO: 47) |
| 341F/P | GAATGANNNNCCTACGGGAGGCAG | 518R/p | cttgtaATTACCGCGGCTGCTGG |
| | CAG (SEQ ID NO: 48) | | (SEQ ID NO: 49) |
| 341F/Q | GCGCCANNNNCCTACGGGAGGCAG | 518R/q | aagcgaATTACCGCGGCTGCTG |
| | CAG (SEQ ID NO: 50) | | G (SEQ ID NO: 51) |
| 341F/R | CTCTACNNNNCCTACGGGAGGCAG | 518R/r | tcattcATTACCGCGGCTGCTGG |
| | CAG (SEQ ID NO: 52) | | (SEQ ID NO: 53) |
| 341F/S | GGTTTCNNNNCCTACGGGAGGCAG | 518R/s | tggcgcATTACCGCGGCTGCTG |
| | CAG (SEQ ID NO: 54) | | G (SEQ ID NO: 55) |
| 341F/T | TAAGGCNNNNCCTACGGGAGGCAG | 518R/t | aaggacATTACCGCGGCTGCTG |
| | CAG (SEQ ID NO: 56) | | G (SEQ ID NO: 57) |
| 341F/U | TCGGGANNNNCCTACGGGAGGCAG | 518R/u | atcctaATTACCGCGGCTGCTG |
| | CAG (SEQ ID NO: 58) | | G (SEQ ID NO: 59) |
| 341F/V | TTCGAANNNNCCTACGGGAGGCAG | 518R/v | cactcaATTACCGCGGCTGCTG |
| | CAG (SEQ ID NO: 60) | | G (SEQ ID NO: 61) |
| 341F/W | GCGGACNNNNCCTACGGGAGGCAG | 518R/w | ccgcaaATTACCGCGGCTGCTG |
| | CAG (SEQ ID NO: 62) | | G (SEQ ID NO: 63) |
| 341F/X | ATTGGCNNNNCCTACGGGAGGCAG | 518R/x | aaaaccATTACCGCGGCTGCTG |
| | CAG (SEQ ID NO: 64) | | G (SEQ ID NO: 65) |
| 341F/Y | TTATTCNNNNCCTACGGGAGGCAG | 518R/y | gccttaATTACCGCGGCTGCTG |
| | CAG (SEQ ID NO: 66) | | G (SEQ ID NO: 67) |
| 341F/Z | TGGAGCNNNNCCTACGGGAGGCAG | 518R/z | tcccgaATTACCGCGGCTGCTG |
| | CAG (SEQ ID NO: 68) | | G (SEQ ID NO: 69) |
| 341F/AA | CTTCGANNNNCCTACGGGAGGCAG | 518R/aa | ttcgaaATTACCGCGGCTGCTG |
| | CAG (SEQ ID NO: 70) | | G (SEQ ID NO: 71) |
| 341F/AB | GGAGAANNNNCCTACGGGAGGCAG | 518R/ab | gtccgcATTACCGCGGCTGCTG |
| | CAG (SEQ ID NO: 72) | | G (SEQ ID NO: 73) |
| 341F/AC | TTTCACNNNNCCTACGGGAGGCAG | 518R/ac | aaagcaATTACCGCGGCTGCTG |
| | CAG (SEQ ID NO:74) | | G (SEQ ID NO: 75) |
| 341F/AD | TCCGTCNNNNCCTACGGGAGGCAG | 518R/ad | agaagaATTACCGCGGCTGCTG |
| | CAG (SEQ ID NO: 76) | | G (SEQ ID NO: 77) |
| 341F/AE | TGTGCCNNNNCCTACGGGAGGCAG | 518R/ae | gaataaATTACCGCGGCTGCTG |
| | CAG (SEQ ID NO: 78) | | G (SEQ ID NO: 79) |
| 341F/AF | TGCCGANNNNCCTACGGGAGGCAG | 518R/af | gctccaATTACCGCGGCTGCTG |
| | CAG (SEQ ID NO: 80) | | G (SEQ ID NO: 81) |
| 341F/AG | GGCCACNNNNCCTACGGGAGGCAG | 518R/ag | ttctccATTACCGCGGCTGCTGG |
| | CAG (SEQ ID NO: 82) | | (SEQ ID NO: 83) |
| 341F/AH | TATATCNNNNCCTACGGGAGGCAG | 518R/ah | gtgaaaATTACCGCGGCTGCTG |
| | CAG (SEQ ID NO: 84) | | G (SEQ ID NO: 85) |
| 341F/AI | CAGGCCNNNNCCTACGGGAGGCAG | 518R/ai | cagatcATTACCGCGGCTGCTG |
| | CAG (SEQ ID NO: 86) | | G (SEQ ID NO: 87) |
| 341F/AJ | GGTAGANNNNCCTACGGGAGGCAG | 518R/aj | aaatgcATTACCGCGGCTGCTG |
| | CAG (SEQ ID NO: 88) | | G (SEQ ID NO: 89) |
| 341F/AK | CGAAACNNNNCCTACGGGAGGCAG | 518R/ak | acaaacATTACCGCGGCTGCTG |
| | CAG (SEQ ED NO: 90) | | G (SEQ ID NO: 91) |

Each 50-μL PCR reaction contained 22 uL water, 25 uL Top Taq Master Mix, 0.5 uL of each forward and reverse barcoded primer, and 2 μL template DNA. The PCR program consisted of an initial DNA denaturation step at 95° C. for (5 min), 25 cycles of DNA denaturation at 95° C. (1 min), an annealing step at 50° C. (1 min), and an elongation step at 72° C. (1 min), and a final elongation step at 72° C. (7 min). Controls without template DNA were included to ensure that no contamination occurred. Amplicons were run on a 2% agarose gel to ensure adequate amplification. Amplicons displaying bands at ˜160 kb were purified using the Illustra GX PCR DNA Purification kit. Purified samples were diluted 1:50 and quantified using PICOGreen (Invitrogen) in the TECAN M200 (excitation at 480 nm and emission at 520 nm).

Illumina Sequencing:

Pooled PCR amplicons were diluted to 20 ng/uL and sequenced at the V3 hyper-variable region using Hi-Seq 2000 bidirectional Illumina sequencing and Cluster Kit v4 (Macrogen Inc., Seoul, Korea). Library preparation was done using TruSeq DNA Sample Prep V2 Kit (Illumina) with 100 ng of DNA sample and QC library by Bioanalyzer DNA 1000 chip (Agilent).

Bioinformatics:

Samples were pre-processed, denoised, and quality filtered by size using Mothur⁵. Representative sequences were clustered into operational taxonomic units (OTUs) using CrunchClust⁶ and classified against the Greengenes Databases according to 97% similarity. Any OTUs present less than 5 times among all samples were removed from the analysis.

Quantitative Polymerase Chain Reaction:

The abundances of specific intestinal bacterial genera were measured in the above 16S rDNA V3 amplicons using group-specific 16S rRNA gene primers for the following genera; Lachnospira, Veillonella, Rothia, Faecalibacterium, and Bifidobacterium (Table 8).

TABLE 8
Quantitative PCT Primer Sequences of Selected Bacterial Genera
qPCR primers

| Taxon targeted | Forward | Reverse |
|---|---|---|
| Eubacteria (all bacteria) | ACT CCT ACG GGA GGC AGC | ATT ACC GCG GCT GCT GGC |
| | AGT (SEQ ID NO: 6) | (SEQ ID NO: 7) |
| Bifidobacterium sp. | CTC CTG GAA ACG GGT GGT | ATA GGA CGC GAC CCC ATC |
| | AAT (SEQ ID NO: 8) | CCA (SEQ ID NO: 9) |
| Veillonella sp. | AAG CTA TCA CTG AAG GAG GG | TCC CAA TGT GGC CGT TCA TCC |
| | (SEQ ID NO: 10) | (SEQ ID NO: 11) |
| Rothia sp. | GCC TGG GAA ACT GGG TCT | CAA GCT GAT AGG CCG TGA G |
| | AAT (SEQ ID NO: 12) | (SEQ ID NO: 13) |
| Faecalibactrium sp. | GGA GCG ATC CGC TTT GAG | AAC CTC TCA GTC CGG CTA CCG |
| | ATG (SEQ ID NO: 14) | A (SEQ ID NO: 15) |
| Lachnospira sp. | GCA ACG CGA AGA ACC TTA CC | ACC ACC TGT CAC CGA TGT TC |
| | (SEQ ID NO: 16) | (SEQ ID NO: 17) |

All AW samples and a randomly selected equal number of control samples were analyzed by quantitative PCR (qPCR). All reactions were carried out in the 7500 Fast Real-Time System (Applied Biosystems, Foster City, Calif.) or the ViiA 7 Real-Time PCR System (Life Technologies Inc., Burlington, ON). Each 10-uL reaction contained 5 uL of IQ SYBR green supermix (Bio-Rad, 5 uL), 0.1 uL of each forward and reverse primer, 0.8 uL of nuclease-free water, and 4 uL of the V3 amplicon. The qPCR program consisted of an initial step at 95° C. (15 min), 40 cycles of 15 s at 94° C., 30 s at 60° C., and 30s at 72° C., and a final cycle of 95° C. at 15 s, 60° C. at 1 min, 95° C. at 15 s, and 60° C. at 15 s. Per primer set, at least two dilutions were run per sample and all dilutions were run in duplicate. Samples were normalized according to the ΔC_{T} method using total 16S rDNA as the reference gene.

PICRUSt:

We used PICRUSt⁸ to generate a profile of putative functions (via metagenome prediction) from our 16S rRNA OTU data. Predicted metagenomes from the same samples analyzed by qPCR were categorized by function at KEGG (Kyoto Encyclopedia of Genes and Genomes) Orthology level 3. To test for significant differences in functional category abundances between AW and control samples, we used the Welch's t test implementation of STAMP⁹. We also tested for differentially abundant metagenomes with DEseq2 ¹⁰ under default settings. The test statistics' p-values were adjusted for multiple testing using the procedure of Benjamini and Hochberg¹¹ (false discovery rate threshold=5%).

Short-Chain Fatty Acid Analysis:

Stool samples were combined with 25% phosphoric acid, vortexed and centrifuged until a clear supernatant was obtained. Supernatants were submitted for GC analysis to the Department of Agricultural, Food and Nutritional Science of the University of Alberta. Only 13 AW samples contained enough material for analysis and 13 additional control samples were randomly selected for this analysis. Samples were analyzed as previously described¹² with modifications. Briefly, samples were combined with 4-methyl-valeric acid as an internal standard and 0.2 ml was injected into the Bruker Scion 456 gas chromatograph, using a Stabilwax-DA 30 m×0.53 mm×0.5 um column (Restek, Bellefonte, Pa.). A standard solution containing acetic acid, proprionic acid, isobutyric acid, butyric acid, isovaleric acid, valeric acid and caproic acid, combined with internal standard was injected in every run.

The PTV injector and FID detector temperatures were held at 250° C. for the entire run. The oven was started at 80° C. and immediately ramped to 210° C. at 45° C./min, where it was held for 5.11 mins. Total run time was 8.00 mins. Helium was used at a constant flow of 20.00 ml/min.

Urine Metabolomics:

250 uL of urine per subject was submitted to Metabolon Inc. (Durham, N.C.) for metabolomics analysis. From the subset of samples selected for qPCR analysis, 16-18 AW and control urine samples were available for metabolomics analysis. Sample preparation was carried out as described previously¹³. Briefly, recovery standards were added prior to the first step in the extraction process for quality control purposes. Proteins were precipitated for removal with methanol under vigorous shaking for 2 min (Glen Mills Genogrinder 2000) followed by centrifugation. The resulting extract was divided into five fractions: one for analysis by ultra high performance liquid chromatography-tandem mass spectrometry (UPLC-MS/MS; positive ionization), one for analysis by UPLC-MS/MS (negative ionization), one for the UPLC-MS/MS polar platform (negative ionization), one for analysis by gas chromatography-mass spectrometry (GC-MS), and one sample was reserved for backup.

The following controls were analyzed with the experimental samples: samples generated from a pool of human urine extensively characterized by Metabolon, Inc. and a cocktail of standards spiked into every analyzed sample, which allowed instrument performance monitoring. Experimental samples and controls were randomized across the platform run.

Mass Spectrometry Analysis

Extracts were subjected to either GC-MS or UPLC-MS/MS. The UPLC-MS/MS platform utilized a Waters Acquity UPLC with Waters UPLC BEH C18-2.1×100 mm, 1.7 μm columns and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in acidic or basic LC-compatible solvents, each of which contained eight or more injection standards at fixed concentrations to ensure injection and chromatographic consistency. Extracts reconstituted in acidic conditions were gradient eluted using water and methanol containing 0.1% formic acid, while the basic extracts, which also used water/methanol, contained 6.5 mM ammonium bicarbonate. A third aliquot was analyzed via negative ionization following elution from a HILIC column (Waters UPLC BEH Amide 2.1×150 mm, 1.7 μm) using a gradient consisting of water and acetonitrile with 10 mM Ammonium Formate. The MS analysis alternated between MS and data-dependent MS² scans using dynamic exclusion, and the scan range was from 80-1000 m/z:

The samples destined for analysis by GC-MS were dried under vacuum desiccation for a minimum of 18 h prior to being derivatized under dried nitrogen using bistrimethyl-silyltrifluoroacetamide. Derivatized samples were separated on a 5% diphenyl/95% dimethyl polysiloxane fused silica column (20 m×0.18 mm ID; 0.18 um film thickness) with helium as carrier gas and a temperature ramp from 60° to 340° C. in a 17.5 min period. All samples were analyzed on a Thermo-Finnigan Trace DSQ fast-scanning single-quadrupole MS using electron impact ionization (EI) and operated at unit mass resolving power. The scan range was from 50-750 m/z.

Compound Identification, Quantification, and Data Curation

Metabolites were identified by automated comparison of the ion features in the experimental samples to a reference library of chemical standard entries that included retention time, molecular weight (m/z), preferred adducts, and in-source fragments as well as associated MS spectra and curated by visual inspection for quality control using software developed at Metabolon¹⁴. Identification of known chemical entities is based on comparison to metabolomic library entries of purified standards. Commercially available purified standard compounds have been acquired and registered into LIMS for distribution to both the UPLC-MS/MS and GC-MS platforms for determination of their detectable characteristics. Peaks were quantified using area-under-the-curve. Raw area counts for each metabolite in each sample were normalized to correct for variation resulting from instrument inter-day tuning differences by the median value for each run-day, therefore, setting the medians to 1.0 for each run. Missing values were imputed with the observed minimum after normalization. All values were further normalized to the osmolality of each sample.

Human Microbiota Model of Experimental Murine Allergic Asthma:

Bacterial Inoculum Preparation and Inoculation:

Frozen feces from one asthmatic child collected at 3 months of age was used to orally inoculate germ-free mice. A fecal slurry was prepared by scraping a frozen piece of fecal material with a sterile scalpel and combining it with 1 ml of PBS reduced with 0.05% of cysteine-HCl to protect anaerobic species. This type of adoptive transfer has been shown to be effective in transferring human microbiota into mice. The sample was vortexed and centrifuged at 3000 g to remove debris. Solid cultures of Faecalibacterium prausnitzii (ATCC 27766), Veillonella parvula (ATCC 10790), Rothia mucilaginosa (ATCC 49040) and Lachnospira multipara (DSM-3073) were grown on Fastidious Anaerobe (FA) agar at 37° C. under anaerobic conditions. One colony of each culture was added to 2 ml liquid FA medium and grown for 24 h. The cell concentrations of the fecal slurry and the FLVR culture were calculated by turbidometry at 600 nm and normalized to OD=0.3 with reduced PBS.

Four female and four male 6-week old germ-free mice (Swiss Webster) were purchased from Taconic (Hudson, N.Y.). Immediately upon arrival, two female and two male mice were randomly selected to be orally gavaged with 50 μl of the fecal slurry (AW), and the remaining mice were inoculated with 40 μl of the same fecal slurry combined with 10 μl of the FLVR culture. Oral gavages with the microbial treatments were repeated on days 3, 7 and 14 post arrival. After the second inoculation mice were paired for mating. To further increase the microbial colonization of the F1 mice with the experimental inocula, the abdominal and nipple area of the mothers was swabbed with the corresponding bacterial preparations on days 3 and 7 after birth.

Experimental Allergic Asthma Model:

Experimental murine allergic asthma was induced in all F1 mice from two subsequent litters for each breeding pair, at 7-8weeks of age, as previously described with minor modifications. Although this model does not fully recapitulate the phenotype of human allergic asthma it is a useful model for evaluating many aspects of this lung inflammatory disease. No statistical methods were used to estimate sample size in animal experiments. A total of 8 control, 18 AW and 28 AW+FLVR mice were used in the two combined experiments. Mice were sensitized intraperitoneally with 10 μg grade V OVA and 1.3 mg aluminum hydroxide (both from Sigma) on days 0 and 7. On days 21, 22, 23, and 24, mice were challenged intranasally with 50 μg of LPS-free OVA in PBS, and on days 25 and 26 with 100 μg of grade V OVA (Sigma). On days 27, mice were anaesthetized with 200 mg kg-1 ketamine and 10 mg kg-1 xylazine and blood was collected by cardiac puncture. After sacrifice, BALs were performed by 3×1 ml washes with PBS. Total BAL counts were blindly assessed by counting cells in a hemocytometer. Eosinophils, neutrophils, macrophages and lymphocytes were quantified from cytospins (Thermo Shandon, Pittsburg, Pa.) stained with HemaStain (Fisher Scientific), based on standard morphological criteria. All protocols used in these experiments were approved by the Animal Care Committee of the University of British Columbia.

Determination of Serum OVA-Specific Igs:

OVA specific IgE, IgG1, and IgG2a in serum were measured by enzyme-linked immunosorbent assay (Chondrex, Redmond, Wash.).

Histology:

Lungs were collected and fixed in 10% formalin, embedded in paraffin, cut longitudinally into 5-μm sections and stained with haematoxylin and eosin. Inflammation was blindly assessed from five fields per section, each graded on a scale of 1-5 (1=no signs of disease, 5=severe disease) for each of the following parameters: (1) peribronchial infiltration, (2) perivascular infiltration, (3) parenchymal infiltration and (4) epithelium damage for a maximum score of 25.

Cytokines:

Lung tissue homogenates were centrifuged twice at 16,000 g, and the supernatants were stored at −80° C. The levels of IL-2, IL-4, IL-6, IL-10, TNF, IFN-γ, and IL-17A were determined using the Cytometric Bead Array (CBA) assay Th1/Th2/Th17 kit (Catalog #560485 BD Biosciences, Ontario, Canada). Levels of IL-5, IL-9 and IL-13 were determined by CBA flex set (Catalog #558302, 558348 and 558349, BD Biosciences, Ontario, Canada) according to the manufacturers' instructions. Cytokine concentrations were normalized to protein concentration calculated by the Bradford assay (Sigma). IL-9 and IL-13 analysis did not yield results above the sensitivity limit of the assay.

Statistical Analysis:

An exact logistic regression model based on Markov Chain Monte Carlo (MCMC) sampling¹⁷⁻¹⁹ was developed and odds ratios (ORs) were used to evaluate the risk associated with the AW group according to specific clinical data. ORs and the adjusted lower and upper confidence intervals were calculated according to the following formula e^{(ln(OR))} and e^{(ln(CI)}, respectively. ln(CI) is equal to the exact upper and lower confidence intervals in extended data table 1. Only subjects for which all the data were available were included in the model (n_{AW}=21, n_{control}=74). We assessed fecal microbial diversity and the relative abundance of bacterial taxa using Phyloseq²⁰ along with additional R-based computational tools²¹⁻²⁶ in R-studio (R-Studio, Boston, Mass.). Principal components analysis (PCA) was conducted using MetaboAnalyst²⁷⁻²⁸ and statistically confirmed by pennanova²⁰. The Shannon diversity index was calculated using Phyloseq and statistically confirmed by Mann-Whitney (GraphPad Prism software, version 5c, San Diego, Calif.). The ‘mt’ function in Phylsoeq²⁰ was used to calculate multi-inference-adjusted P-values to identify differentially abundant OTUs between the 3-month and 1-year samples and among the four phenotypes; AW, atopy only, wheeze only, and controls. Differences between the control and AW groups were determined by Mann-Whitney for qPCR. All SCFAs and urine metabolites were subject to the Shapiro-Wilk test for normality and differences between control and AW groups were determined by t-test (glycocholenate sulfate and glycohyocholate) or Mann-Whitney Wilcoxon. No human samples were excluded from statistical tests. For analyses using human samples, the F-test found no significant differences between the variances of the groups. Differences between AW, AW+FLVR and naive groups in mice experiments were determined by ANOVA for BAL counts, BAL cell differentials, histology scoring, lung cytokine and serum immunoglobulin concentrations. All data points in graphs represent biological replicates. Outliers were detected and excluded from mouse experiment data only, using the ROUT method (Q=1%) in Graph Pad Prism. Statistical significance was defined as P<0.05.

Results

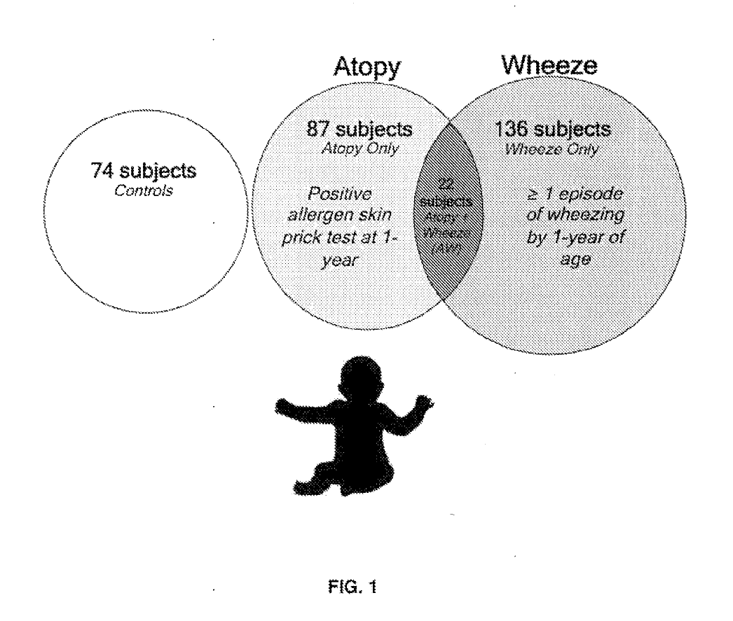

We selected 319 children from this cohort for gut microbiome analysis, as set out herein, grouped into four clinically-distinct phenotypes based on allergy skin prick testing (i.e. atopy) and clinical data (i.e. wheeze) at age 1-year: atopy+wheeze (AW, n=22), atopy only (n=87), wheeze only (n=136), and controls (n=74) (FIG. 1). In addition, 2 and 3-year clinical data was used to apply the stringent Asthma Predictive Index (API), a clinically-validated predictive index for the presence of active asthma at school age⁷, to confirm the clinical significance of these 1-year phenotypes. A positive stringent API at 3-years of age is associated with a 77% chance of active asthma between ages 6 and 13-years⁸. CHILD study subjects in the AW group at 1-year of age were 13.5 times more likely than the control group [95% CI: 3.2 to 57.4] to have a positive stringent API (FIG. 1). Compared to atopy only and wheeze only phenotypes, the AW group was also significantly enriched in the positive API category, identifying these children as the most at risk for active asthma at school age.

In line with other asthma epidemiologic studies⁹, exact logistic regression analysis identified antibiotic exposure in the first year of life [OR: 5.6, p=0.009] and atopic dermatitis at 1-year [OR: 6.4, p=0.005] as factors that increased a subject's risk of being classified in the AW group compared to controls (Table 1).

TABLE 1
Characteristics of the study population.

| | | 1-year Phenotype | | | | 95% Confidence | | |
|---|---|---|---|---|---|---|---|---|
| | | Atopy + | | | | Interval (CI) | | |
| | | Wheeze | | Control | *Odds Ratio (OR) | Lower | Upper | P-value |
| Antibiotic Exposure | 1 or more | n = 9 | (42%) | n = 12 (16%) | OR = 5.6, antibiotics in | 1.3 | 81 | 0.009 |
| (from birth to | None | n = 12 | (58%) | n = 62 (84%) | the first year of life | | | |
| 1-year of age) | Total (100%) | n = 21 | | n = 74 | increase odds of | | | |
| | | | | | developing atopy and | | | |
| | | | | | wheeze | | | |
| Atopic Dermatitis | Yes | n = 13 | (62%) | n = 18 (24%) | OR = 6.4, AD/eczema | 1.5 | 67 | 0.005 |
| (AD) or Eczema at | No | n = 8 | (38%) | n = 56 (76%) | diagnosis (at 1-year) | | | |
| 1-year | Total (100%) | n = 21 | | n = 74 | increases odds of | | | |
| | | | | | developing atopy and | | | |
| | | | | | wheeze | | | |
| Atopic Dermatitis | Yes | n = 5 | (24%) | n = 4 (5%) | OR = 2.2 | 0.1 | 18.2 | 0.53 |
| (AD) or Eczema at | No | n = 16 | (76%) | n = 70 (95%) | | | | |
| 3-months | Total (100%) | n = 21 | | n = 74 | | | | |
| Antibiotic Exposure | 1 or more | n = 0 | (0%) | n = 4 (5%) | OR = 0.33 | Undef. * | 3.3 | 0.24 |
| (from birth to | None | n = 21 | (100%) | n = 70 (95%) | | | | |
| 3-months of age) | Total (100%) | n = 21 | | n = 74 | | | | |
| Sex | Female | n = 7 | (33%) | n = 38 (51%) | OR = 0.38 | 0.07 | 1.4 | 0.15 |
| | Male | n = 14 | (67%) | n = 36 (49%) | | | | |
| | Total (100%) | n = 21 | | n = 74 | | | | |
| Delivery Mode | Vaginal | n = 16 | (76%) | n = 58 (78%) | OR = 0.74 | 0.15 | 4.1 | 1 |
| | Caesarean | n = 5 | (24%) | n = 16 (23%) | | | | |
| | Total (100%) | n = 21 | | n = 74 | | | | |
| Feeding Methods | Breast-fed | n = 15 | (71%) | n = 60 (81%) | OR = 0.5 | 0.07 | 4.1 | 0.69 |
| (at 3-months) | Not breast-fed | n = 6 | (29%) | n = 14 (19%) | | | | |
| | Total (100%) | n = 21 | | n = 74 | | | | |
| Feeding Methods | Breast-fed | n = 7 | (33%) | n = 25 (34%) | OR = 1.2 | 0.2 | 6.7 | 0.73 |
| (at 1-year) | Not breast-fed | n = 14 | (67%) | n = 49 (66%) | | | | |
| | Total (100%) | n = 21 | | n = 74 | | | | |
| M.D. Diagnosed | Yes | n = 3 | (14%) | n = 10 (14%) | OR = 1.1 | 0.1 | 7.4 | 1 |
| Paternal Asthma | No | n = 18 | (86%) | n = 64 (86%) | | | | |
| | Total (100%) | n = 21 | | n = 74 | | | | |
| M.D. Diagnosed | Yes | n = 7 | (33%) | n = 24 (32%) | OR = 1.25 | 0.3 | 6 | 1 |
| Maternal Asthma | No | n = 14 | (67%) | n = 50 (68%) | | | | |

| | | |
|---|---|---|
| Total (100%) | n = 21 | n = 74 |

*The group listed first for each variable (i.e. 1 or more for antibiotic exposure) is the reference group for interpreting the odds ratio.
* A finite lower bound for the confidence interval could not be obtained because the observed value of the sufficient statistic is the maximum possible value.

Caesarean birth¹⁰, exclusive formula feeding¹⁰, and antibiotic exposure¹¹ in infancy are also common factors associated with gut microbial dysbiosis, which prompted their inclusion in our assessment of the 3-month and 1-year fecal microbiota, but these were not significant factors in this sub-population.

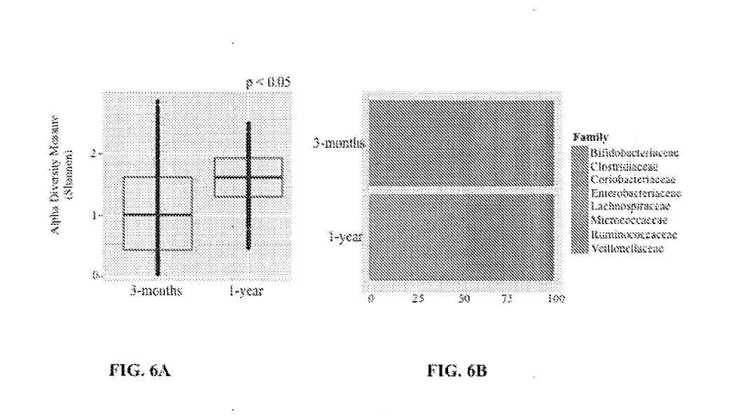

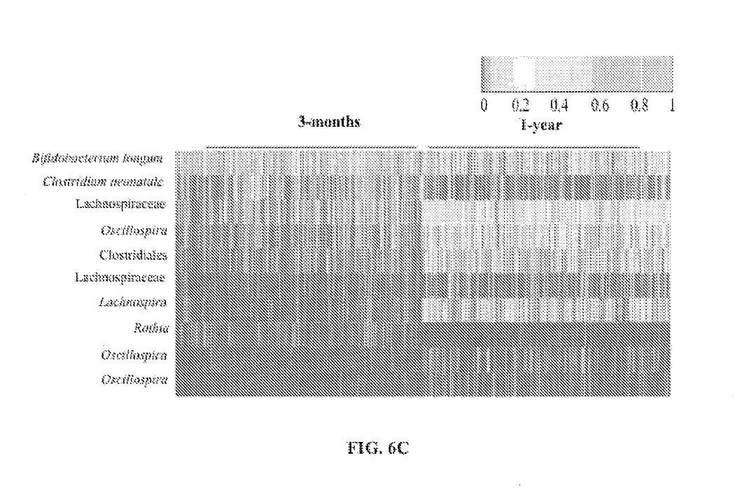

Consistent with microbiome studies in other cohorts of young children¹², principal component analysis (PCA) identified age as the main driver of microbial and metabolic shifts in this cohort (FIGS. 5 and 6 and Table 2).

TABLE 2
Differentially abundant OTUs between 3-month and 1-year samples.*

| Operational | | Median | Median | | | | | | | |
|---|---|---|---|---|---|---|---|---|---|---|
| Taxonamic | Adjusted | Abundance | Abundance | | | | | | | |
| Unit (OTU) | P value | 3-months | 1-year | Kingdom | Phylum | Class | Order | Family | Genus | Species |
| 1 | 0.0019 | 0.6 | 0.1 | Bacteria | Actinobacteria | Actinobacteria | Bifidobacteriales | Bifidobacteriaceae | Bifidobacterium | longum |
| 4 | 0.0019 | 0.02 | 0.01 | Bacteria | Firmicutes | Clostridia | Clostridiales | Clostridiaceae | Clostridium | neonatale |
| 2 | 0.0019 | 0.04 | 0.2 | Bacteria | Firmicutes | Clostridia | Clostridiales | Lachnospiraceae | | |
| 6 | 0.0019 | 0.01 | 0.06 | Bacteria | Firmicutes | Clostridia | Clostridiales | Ruminococcaceae | Oscillospira | |
| 5 | 0.0019 | 0.01 | 0.05 | Bacteria | Firmicutes | Clostridia | Clostridiales | | | |
| 7 | 0.0289 | 1.00E−03 | 5.00E−03 | Bacteria | Firmicutes | Clostridia | Clostridiales | Lachnospiraceae | | |
| 3 | 0.0019 | 1.00E−03 | 0.09 | Bacteria | Firmicutes | Clostridia | Clostridiales | Lachnospiraceae | Lachnospira | |
| 20 | 0.0019 | 2.00E−03 | 4.00E−04 | Bacteria | Actinobacteria | Actinobacteria | Actinomycetales | Micrococcaceae | Rothtia | |
| 8 | 0.0019 | 2.00E−03 | 1.00E−04 | Bacteria | Proteobacteria | Gammaproteobacteria | Enterobacteriales | Enterobacteriaceae | | |
| 9 | 0.0087 | 8.00E−04 | 6.00E−04 | Bacteria | Firmicutes | Clostridia | Clostridiales | Veillonellaceae | Veillonella | |
| 31 | 0.0019 | 2.00E−03 | 1.00E−04 | Bacteria | | | | | | |
| 10 | 0.0019 | 6.00E−04 | 2.00E−04 | Bacteria | Proteobacteria | Gammaproteobacteria | Enterobacteriales | Enterobacteriaceae | | |
| 12 | 0.0244 | 2.00E−02 | 1.80E−04 | Bacteria | Firmicutes | Clostridia | Clostridiales | Veillonellaceae | Veillonella | |
| 53 | 0.0019 | 2.00E−04 | 2.00E−05 | Bacteria | Actinobacteria | Coriobacteria | Coriobacteriales | Coriobacteriaceae | Atopobium | |
| 32 | 0.0019 | 2.00E−04 | 0 | Bacteria | Firmicutes | Clostridia | Clostridiales | Clostridiaceae | | |
| 13 | 0.0019 | 0 | 2.00E−03 | Bacteria | Firmicutes | Clostridia | Clostridiales | Ruminococcaceae | Oscillospira | |
| 34 | 0.0019 | 0 | 2.00E−04 | Bacteria | | | | | | |
| 39 | 0.0019 | 2.00E−04 | 1.70E−04 | Bacteria | Firmicutes | Clostridia | Clostridiales | | | |
| 38 | 0.0019 | 8.00E−05 | 0 | Bacteria | Firmicutes | Clostridia | Clostridiales | Clostridiaceae | | |
| 47 | 0.0019 | 4.00E−04 | 1.00E−04 | Bacteria | | | | | | |
| 113 | 0.0019 | 2.00E−04 | 4.00E−05 | Bacteria | Actinobacteria | Actinobacteria | Bifidobacteriales | Bifidobacteriaceae | Bifidobacterium | longum |
| 15 | 0.0019 | 2.00E−05 | 1.00E−03 | Bacteria | Firmicutes | Clostridia | Clostridiales | Lachnospiraceae | | |
| 16 | 0.0019 | 0 | 2.00E−03 | Bacteria | Firmicutes | Clostridia | Clostridiales | Ruminococcaceae | Faecalibacterium | |
| 48 | 0.0019 | 4.20E−05 | 1.40E−05 | Bacteria | Actinobacteria | | | | | |
| 14 | 0.0019 | 0 | 1.00E−03 | Bacteria | Firmicutes | Clostridia | Clostridiales | Ruminococcaceae | Oscillospira | |
| 30 | 0.0019 | 0 | 4.00E−05 | Bacteria | Firmicutes | Clostridia | Clostridiales | Ruminococcaceae | Oscillospira | |
| 156 | 0.0019 | 8.00E−05 | 0 | Bacteria | Actinobacteria | Actinobacteria | Bifidobacteriales | Bifidobacteriaceae | Bifidobacterium | longum |
| 49 | 0.0019 | 2.00E−04 | 1.00E−04 | Bacteria | | | | | | |
| 72 | 0.0019 | 2.00E−04 | 0 | Bacteria | Proteobacteria | Gammaprcteobacteria | | | | |
| 70 | 0.0019 | 8.00E−05 | 0 | Bacteria | | | | | | |
| 64 | 0.0019 | 9.00E−05 | 4.00E−05 | Bacteria | | | | | | |
| 24 | 0.0075 | 0 | 4.00E−05 | Bacteria | Proteobacteria | Alphaproteobacteria | RF32 | | | |
| 68 | 0.0019 | 4.00E−05 | 6.00E−05 | Bacteria | | | | | | |
| 84 | 0.0428 | 4.50E−05 | 5.00E−05 | Bacteria | | | | | | |
| 94 | 0.0019 | 3.50E−05 | 3.60E−05 | Bacteria | | | | | | |
| 11 | 0.0019 | 0 | 2.00E−04 | Bacteria | Firmicutes | Clostridia | Clostridiales | Lachnospiraceae | | |
| 56 | 0.0419 | 4.20E−05 | 5.00E−05 | Bacteria | Firmicutes | Clostridia | Clostridiales | Lachnospiraceae | | |
| 29 | 0.0019 | 0 | 1.00E−04 | Bacteria | Firmicutes | Clostridia | Clostridiales | | | |
| 23 | 0.0019 | 0 | 8.00E−05 | Bacteria | Firmicutes | Clostridia | Clostridiales | | | |
| 27 | 0.0019 | 0 | 9.00E−05 | Bacteria | Firmicutes | Clostridia | Clostridiales | | | |
| 40 | 0.0019 | 0 | 3.00E−04 | Bacteria | Firmicutes | Clostridia | Clostridiales | Lachnospiraceae | | |
| 42 | 0.0019 | 0 | 2.00E−04 | Bacteria | Firmicutes | Clostridia | Clostridiales | Lachnospiraceae | | |

*OTUs in order of greatest difference between 3-month and 1-year median abundance

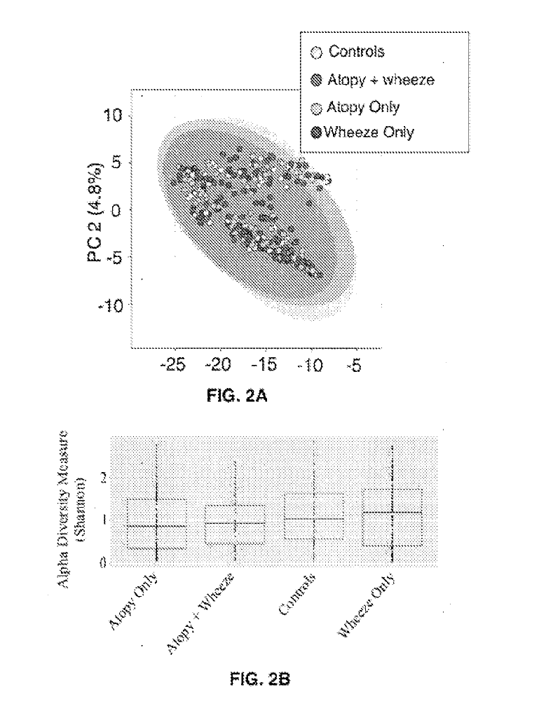

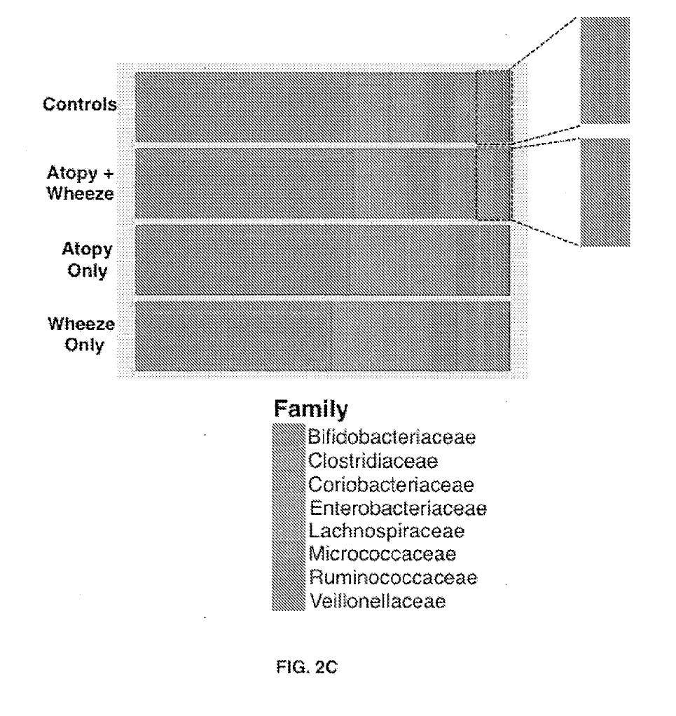

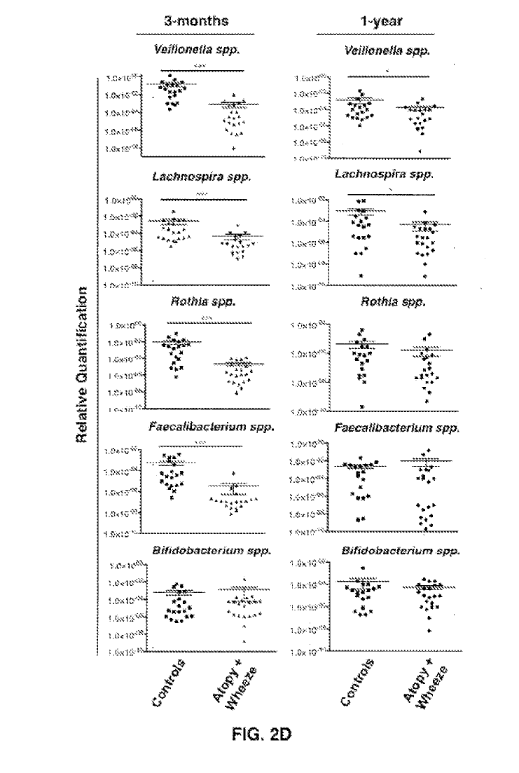

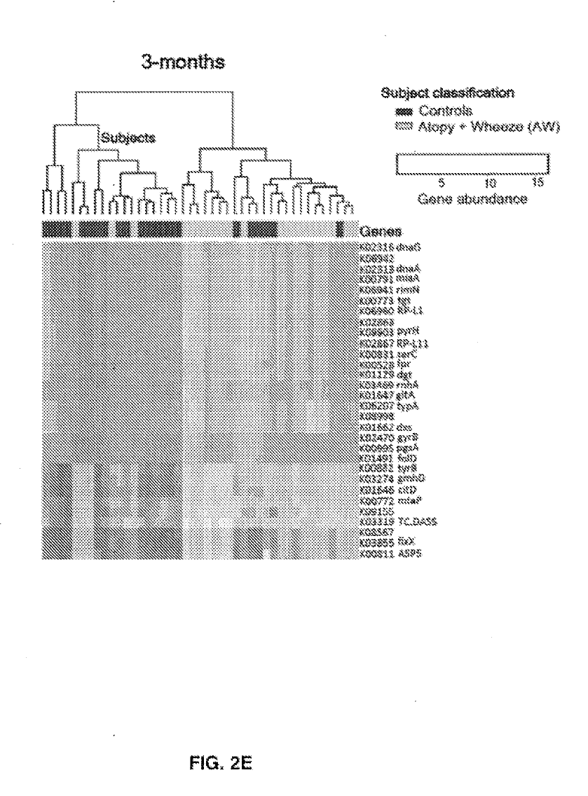

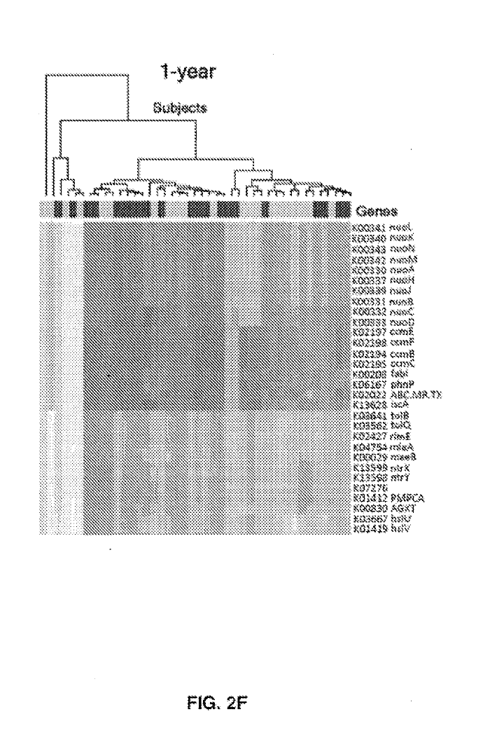

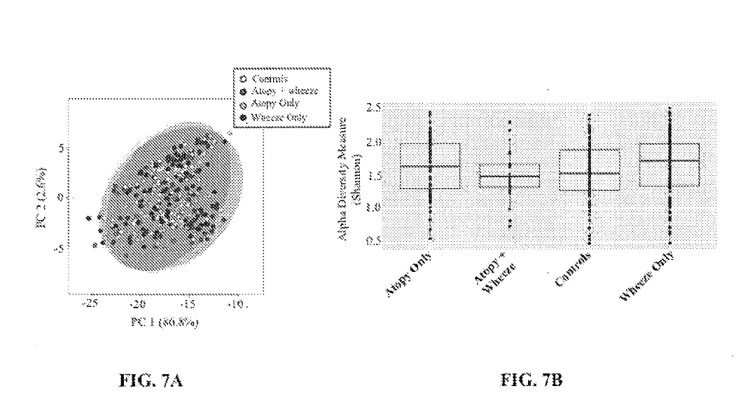

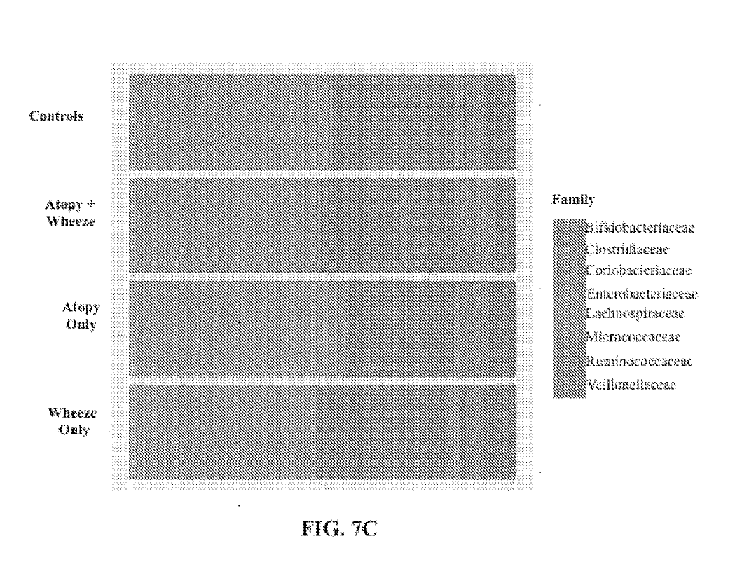

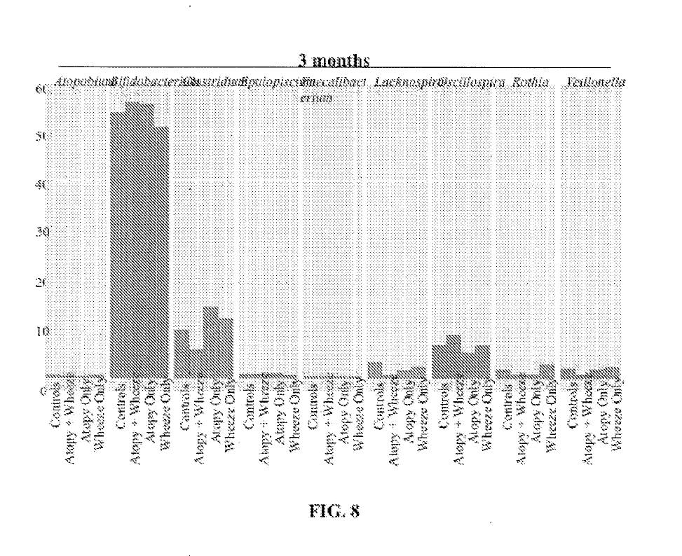

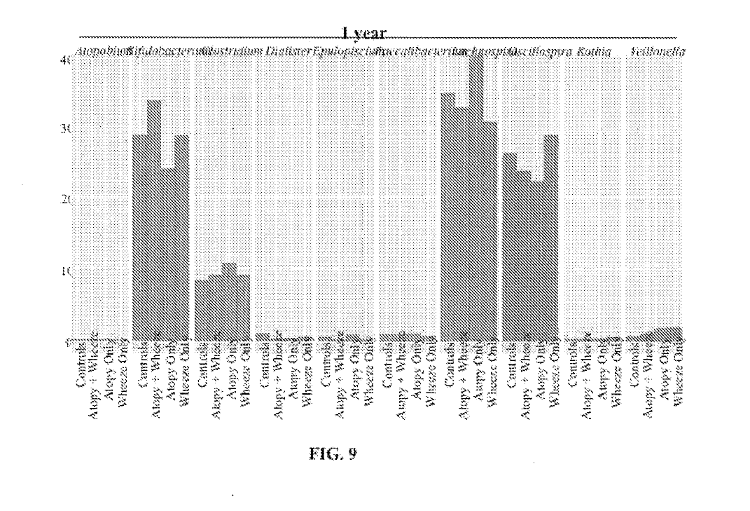

Overall gut community composition did not differ substantially among clinical phenotypes, as shown by PCA of the 3-month and 1-year samples (FIG. 2A, FIG. 7A). A non-significant decrease in diversity in the AW group compared to controls at 3-months and 1-year (FIG. 2B, FIG. 7B) was observed. Nevertheless, a comparison of relative taxa abundance according to the clinical phenotype (FIG. 2C) identified differences in the prevalence of some less abundant bacterial taxa (i.e. Microccocaceae and Veillonellaceae) in the 3-month stool samples, differences which were not present at 1-year (FIG. 7C). These differences were even more apparent at the genus level (FIGS. 8 and 9), where the AW group exhibited lower abundance of the genera Faecalibacterium, Lachnospira, Rothia, and Veillonella exclusively at 3-months. Statistical analysis of the top 50 OTUs across phenotypes yielded 8 differentially abundant OTUs at 3-months and only one at 1-year (Table 3; mt test, raw p<0.05).

TABLE 3
Differentially abundant taxa among the four clinical phenotypes at 3 months and 1 year.

| | OTU# | Raw p | Adjusted p | Domain | Phylum | Class | Order |
|---|---|---|---|---|---|---|---|
| 3-months | Otu00007 | 0.0043 | 0.1784 | Bacteria | Firmicutes | Clostridia | Clostridiales |
| | Otu00005 | 0.0075 | 0.1986 | Bacteria | Firmicutes | Clostridia | Clostridiales |
| | Otu00009 | 0.008 | 0.2033 | Bacteria | Firmicutes | Clostridia | Clostridiales |
| | Otu00012 | 0.0084 | 0.3244 | Bacteria | Firmicutes | Clostridia | Clostridiales |
| | Otu00047 | 0.0108 | 0.392 | Bacteria | NA | NA | NA |
| | Otu00016 | 0.0111 | 0.3934 | Bacteria | Firmicutes | Clostridia | Clostridiales |
| | Otu00092 | 0.0323 | 0.7387 | Bacteria | Firmicutes | Clostridia | Clostridiales |
| | Otu00020 | 0.0499 | 0.8741 | Bacteria | Actinobacteria | Actinobacteria | Actinotmycetales |
| 1-year | Otu00006 | 0.0302 | 0.686 | Bacteria | Firmicutes | Clostridia | Clostridiales |

| | Family | Genus | Species |
|---|---|---|---|
| 3-months | Lachnospiraceae | Lachnospira | NA |
| | NA | NA | NA |
| | Veillonellaceae | Veillonella | NA |
| | Veillonellaceae | Veillonella | NA |
| | NA | NA | NA |
| | Ruminococcaceae | Faecalibacterium | NA |
| | Peptostreptococcaceae | Peptostreptococcus | anaerobius |
| | Micrococcaceae | Rothia | NA |
| 1-year | Ruminococcaceae | Oscillospira | NA |

To validate these results, a subset of samples (n_{AW}=21, n_{CTRL}=20) was used to determine the abundance of the genera, Veillonella, Lachnospira, Rothia, Faecalibacterium, and Bifidobacterium by quantitative PCR (qPCR). Again, qPCR revealed significantly lower abundances of Veillonella, Lachnospira, Rothia, and Faecalibacterium in the 3-month AW stool samples (FIG. 2D, Mann-Whitney P<0.001). Consistent with the 16S sequencing results, these differences were much less apparent in the 1-year stool (Veillonella and Lachnospira showed less significant differences (P<0.05) whereas the other 3 genera were not significantly different), indicating that a lower abundance of these bacterial taxa before infants reach 3-months of age is associated with atopy and wheezing at 1-year of age. Moreover, as the children in the AW phenotype are 13.5 times more likely to have a positive API than the control group (FIG. 1), our results also suggest that lower abundances of these taxa in early life is associated with a high risk of active asthma at school age.

To determine the importance of this early-life dysbiosis in the infants at highest risk of asthma, the functional potential of the fecal microbiota was predicted using PICRUSt, an algorithm that infers the functional metagenome of microbial communities based on marker gene data and reference bacterial genomes. We compared the inferred genetic composition of the fecal microbiota in the same subset of samples selected for qPCR (FIGS. 2E-F). We observed a number of genes that were associated with the AW phenotype. Of the total 6911 genes (defined as KEGG orthologs; KO) surveyed, 2364 genes were significantly different at 3-months, and only 125, at 1-year (Wald test and FDR, p<0.05). The top 30 differential genes (based on lowest p values; Table 4) highlight their capacity to discriminate between the AW group and controls at 3-months (FIG. 2E), but not at 1-year of age (FIG. 2F).

TABLE 4
PICRUSt-predicted top 30 differential KOs (based on p value) in AW and controls at 3 months and 1 year.

| | | |
|---|---|---|
| KO | Gene | |
| number | name | Definition |

3-months

| | | |
|---|---|---|
| K02316 | dnaG | DNA primase[EC: 2.7.7.-] |
| K06942 | | hypothetical protein |
| K02313 | dnaA | chromosomal replication initiator protein |
| K00791 | miaA | tRNA dimethylallyltransferase [EC: 2.5.1.75] |
| K06941 | rlmN | 23S rRNA (adenine2503-C2)-methyltransferase [EC: 2.1.1.192] |
| K00773 | tgt | queuine tRNA-ribosyltransferase [EC: 2.4.2.29] |
| K06960 | RP-L1 | large subunit ribosomal protein L1 |
| K02863 | | hypothetical protein |
| K09903 | pyrH | uridylate kinase [EC: 2.7.4.22] |
| K02867 | RP-L11 | large subunit ribosomal protein L11 |
| K00831 | serC | phosphoserine aminotransferase [EC: 2.6.1.52] |
| K00528 | fpr | ferredoxin--NADP+ reductase [EC: 1.18.1.2] |
| K01129 | dgt | dGTPase [EC: 3.1.5.1] |
| K03469 | rnhA | ribonuclease HI [EC: 3.1.26.4] |
| K01647 | gltA | citrate synthase [EC: 2.3.3.1] |
| K06207 | typA | GTP-binding protein |
| K08998 | | hypothetical protein |
| K01662 | dxs | 1-deoxy-D-xylulose-5-phosphate synthase [EC: 2.2.1.7] |
| K02470 | gyrB | DNA gyrase subunit B [EC: 5.99.1.3] |
| K00995 | pgsA | CDP-diacylglycerol--glycerol-3-phosphate 3-phosphatidyltransferase [EC: 2.7.8.5] |
| K01491 | folD | methenyltetrahydrofolate cyclohydrolase [EC: 1.5.1.5 3.5.4.9] |
| K00832 | tyrB | aromatic-amino-acid transaminase [EC: 2.6.1.57] |
| K03274 | gmhD | ADP-L-glycero-D-manno-heptose 6-epimerase [EC: 5.1.3.20] |
| K01646 | citD | citrate lyase subunit gamma (acyl carrier protein) |
| K00772 | mtaP | 5′-methylthioadenosine phosphorylase [EC: 2.4.2.28] |
| K09155 | | hypothetical protein |
| K03319 | TC.DASS | divalent anion: Na+ symporter, DASS family |
| K08567 | | hypothetical protein |
| K03855 | fixX | ferredoxin like protein |
| K00811 | ASP5 | aspartate aminotransferase, chloroplastic [EC: 2.6.1.1] |

1-year

| | | |
|---|---|---|
| K00341 | nuoL | NADH-quinone oxidoreductase subunit L [EC: 1.6.5.3] |
| K00340 | nuoK | NADH-quinone oxidoreductase subunit K [EC: 1.6.5.3] |
| K00343 | nuoN | NADH-quinone oxidoreductase subunit N [EC: 1.6.5.3] |
| K00342 | nuoM | NADH-quinone oxidoreductase subunit M [EC: 1.6.5.3] |
| K00330 | nuoA | NADH-quinone oxidoreductase subunit A [EC: 1.6.5.3] |
| K00337 | nuoH | NADH-quinone oxidoreductase subunit H [EC: 1.6.5.3] |
| K00339 | nuoJ | NADH-quinone oxidoreductase subunit J [EC: 1.6.5.3] |
| K00331 | nuoB | NADH-quinone oxidoreductase subunit B [EC: 1.6.5.3] |
| K00332 | nuoC | NADH-quinone oxidoreductase subunit C [EC: 1.6.5.3] |
| K00333 | nuoD | NADH-quinone oxidoreductase subunit D [EC: 1.6.5.3] |
| K02197 | ccmE | cytochrome c-type biogenesis protein CcmE |
| K02198 | ccmF | cytochrome c-type biogenesis protein CcmF |
| K02194 | ccmB | heme exporter protein B |
| K02195 | ccmC | heme exporter protein C |
| K00208 | fabI | enoyl-[acyl-carrier protein] reductase I [EC: 1.3.1.9 1.3.1.10] |
| K06167 | phnP | phosphoribosyl 1,2-cyclic phosphate phosphodiesterase [EC: 3.1.4.55] |
| K02022 | ABC.MR.TX | HlyD family secretion protein |
| K13628 | iscA | iron-sulfur cluster assembly protein |
| K03641 | tolB | TolB protein |
| K03562 | tolQ | biopolymer transport protein TolQ |
| K02427 | rlmE | 23S rRNA (uridine2552-2′-O)-methyltransferase [EC: 2.1.1.166] |
| K04754 | mlaA | phospholipid-binding lipoprotein MlaA |
| K00029 | maeB | malate dehydrogenase (oxaloacetate-decarboxylating)(NADP+) [EC: 1.1.1.40] |
| K13599 | ntrX | two-component system, nitrogen regulation response regulator NtrX |
| K13598 | ntrY | two-component system, nitrogen regulation sensor histidine kinase NtrY [EC: 2.7.13.3] |
| K07276 | | hypothetical protein |
| K01412 | PMPCA | mitochondrial-processing peptidase subunit alpha [EC: 3.4.24.64] |
| K00830 | AGXT | alanine-glyoxylate transaminase [EC: 2.6.1.44 2.6.1.45 2.6.1.51] |
| K03667 | hslU | ATP-dependent HslUV protease ATP-binding subunit HslU |
| K01419 | hslV | ATP-dependent HslUV protease, peptidase subunit HslV [EC: 3.4.25.2] |

This functional difference in the 3-month stool samples suggests potential for the community to influence development of asthma. The functional differences in the AW community involved genes with diverse metabolic functions (i.e. gene replication, carbon metabolism, transporters, amino acid biosynthesis, etc.; Table 5).

TABLE 5
Top 30 biochemical pathways (based on p value) of PICRUSt-predicted KOs in AW and controls at 3 months.
3-months

| | Mean relative frequency (%) | | | p-values |
|---|---|---|---|---|
| Pathway | AW | CTRL | p-values | (corrected) |
| Lipopolysaccharide biosynthesis | 0.025 | 0.050 | 0.001 | 0.044 |
| Lipopolysaccharide biosynthesis proteins | 0.089 | 0.124 | 0.001 | 0.047 |
| Pores ion channels | 0.146 | 0.168 | 0.001 | 0.050 |
| Fluorobenzoate degradation | 0.000 | 0.001 | 0.002 | 0.063 |
| beta-Lactam resistance | 0.001 | 0.002 | 0.002 | 0.063 |
| Biosynthesis and biodegradation of secondary metabolites | 0.010 | 0.016 | 0.002 | 0.063 |
| Chloroalkane and chloroalkene degradation | 0.275 | 0.265 | 0.005 | 0.082 |
| Carbohydrate digestion and | 0.002 | 0.004 | 0.005 | 0.086 |
| absorption | | | | |
| DNA replication | 0.718 | 0.692 | 0.005 | 0.087 |
| Transcription related proteins | 0.001 | 0.002 | 0.005 | 0.090 |
| Mineral absorption | 0.001 | 0.002 | 0.004 | 0.091 |
| Histidine metabolism | 0.608 | 0.590 | 0.007 | 0.093 |
| DNA replication proteins | 1.302 | 1.251 | 0.007 | 0.093 |
| D-Arginine and D-ornithine metabolism | 0.002 | 0.005 | 0.006 | 0.093 |
| Translation proteins | 0.921 | 0.890 | 0.004 | 0.096 |
| Xylene degradation | 0.097 | 0.088 | 0.007 | 0.097 |
| Dioxin degradation | 0.097 | 0.089 | 0.009 | 0.104 |
| Stilbenoid, diarylheptanoid and gingerol biosynthesis | 0.000 | 0.001 | 0.008 | 0.106 |
| Two-component system | 0.960 | 1.146 | 0.010 | 0.112 |
| Chlorocyclohexane and chlorobenzenedegradation | 0.006 | 0.010 | 0.010 | 0.116 |
| Lysine degradation | 0.108 | 0.131 | 0.016 | 0.132 |
| Tryptophan metabolism | 0.141 | 0.163 | 0.017 | 0.134 |
| Phosphatidylinositol signaling system | 0.115 | 0.107 | 0.016 | 0.134 |
| Lysine biosynthesis | 1.003 | 0.957 | 0.016 | 0.137 |
| Valine, leucine and isoleucine degradation | 0.185 | 0.227 | 0.013 | 0.137 |
| Glycan biosynthesis and metabolism | 0.005 | 0.009 | 0.018 | 0.138 |
| Restriction enzyme | 0.197 | 0.179 | 0.014 | 0.139 |

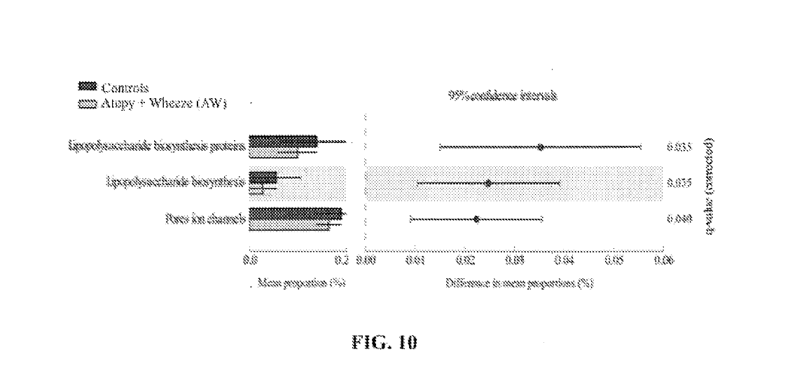

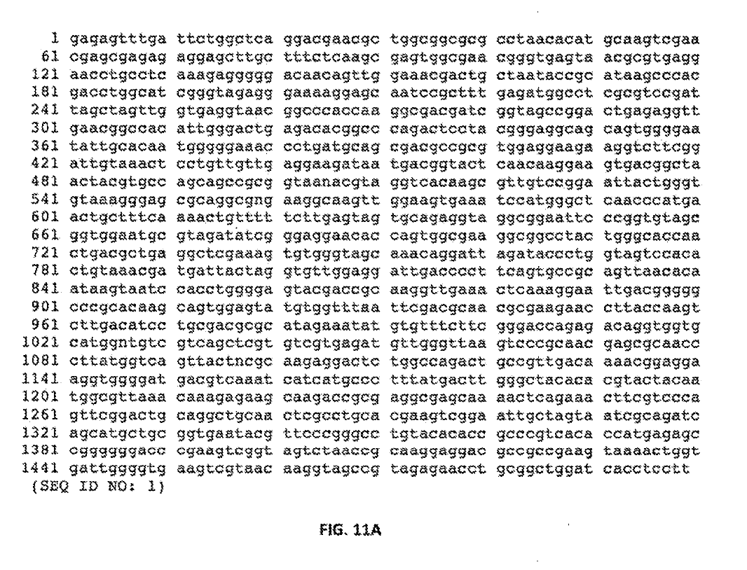

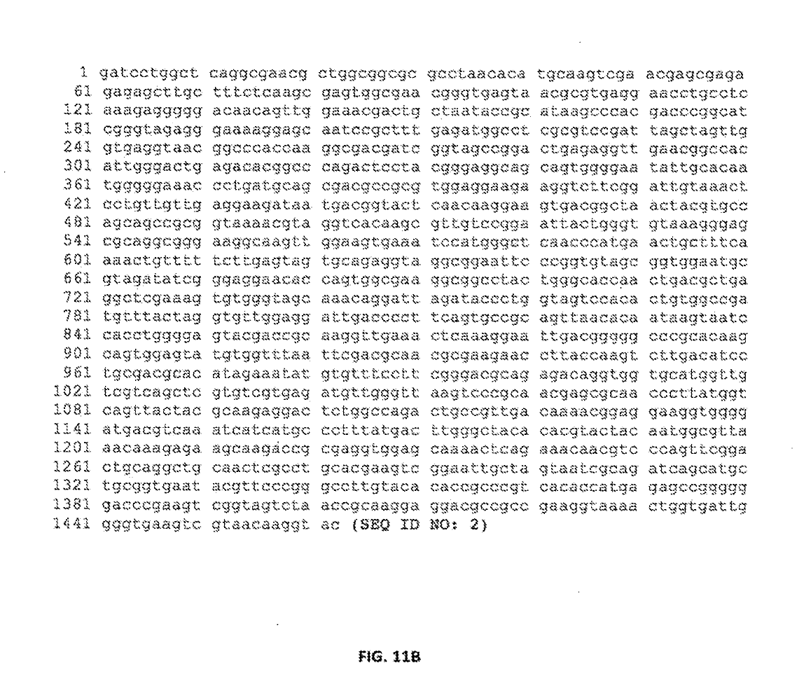

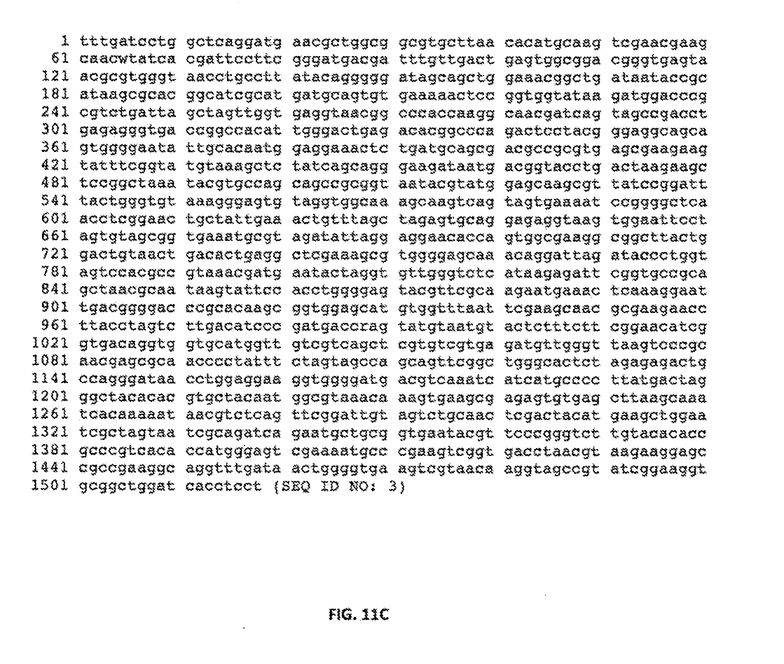

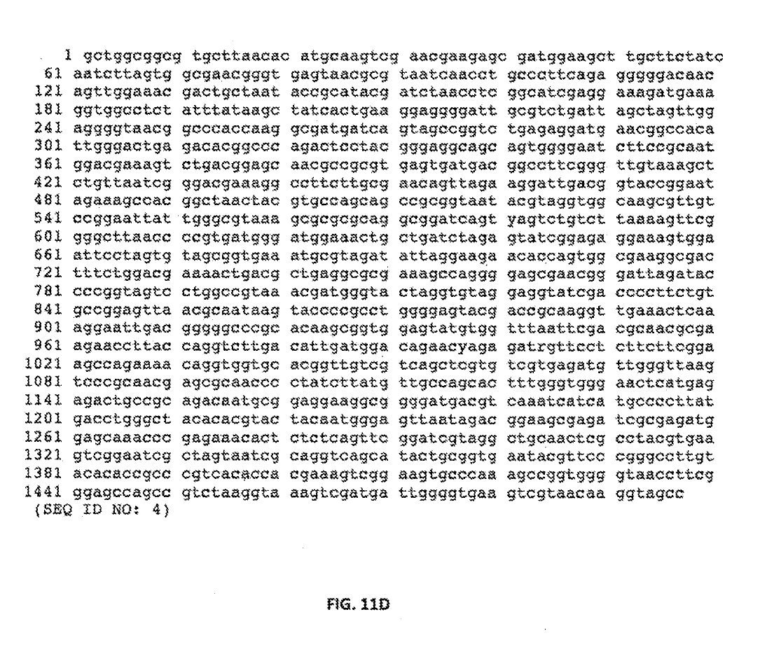

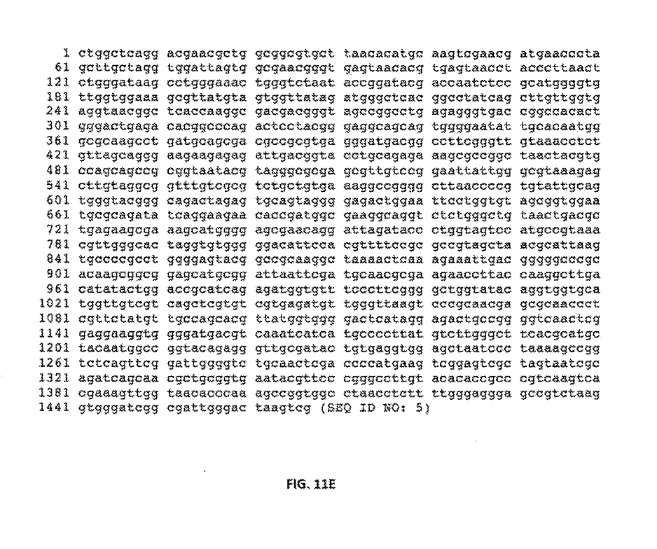

Once these genes were organized into specific metabolic pathways, lipopolysaccharide (LPS) biosynthesis was the pathway differing most between AW and control groups (Welch's t-test, FIG. 10). Once again, significant differences in specific metabolic pathways between the clinical groups were not found in the 1-year samples. Considering that the vast majority of the intestinal bacteria detected at 3-months were Gram positive (all except Enterobacteriaceae and Veillonellaceae), it is possible that the difference in Veillonella species may account for the difference in LPS biosynthesis genes in the AW group.

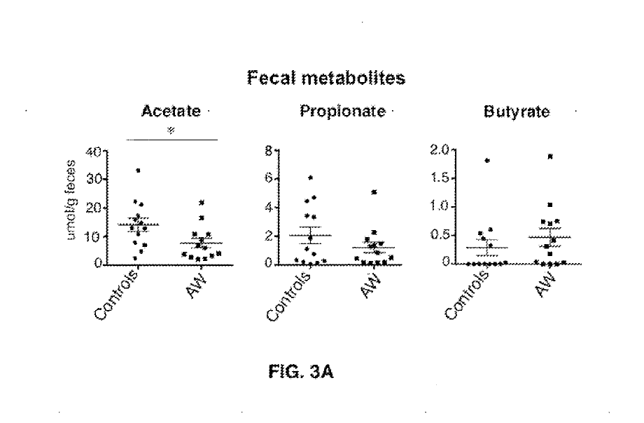

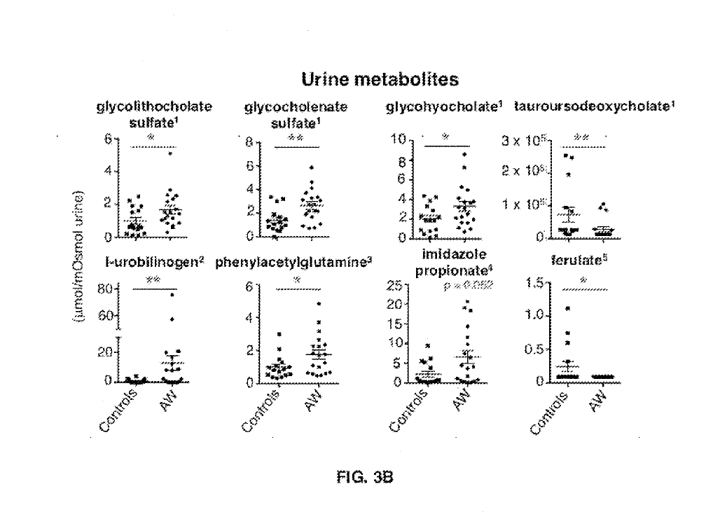

The functional implications of the gut community in AW children were further investigated by measuring SCFA levels in feces and urine, as well by urine metabolomics. At 3-months of age, fecal samples of AW children had a significantly lower concentration of acetate (FIG. 3A and Table 6).

TABLE 6
Short-chain fatty acids in feces and urine in AW and controls at 3 months and 1 year.
SCFA in feces

| | | Acetic Acid | | | Proprionic Acid | | | Isobutyric Acid | | | Butyric Acid | | |
|---|---|---|---|---|---|---|---|---|---|---|---|---|---|
| | | Mean | SD | | Mean | SD | | Mean | SD | | Mean | SD | |
| | Age | (μmol/g feces) | | p | (μmol/g feces) | | p | (μmol/g feces) | | p | (μmol/g feces) | | p |
| Controls | 3 mo | 14.13 | 8.19 | 0.03 | 2.07 | 2.08 | 0.38 | 1.82 | 4.47 | 0.9 | 0.29 | 0.51 | 0.15 |
| AW | 3 mo | 7.77 | 6.01 | | 1.06 | 0.91 | | 0.11 | 0.15 | | 0.47 | 0.56 | |
| Controls | 1 year | 11.96 | 6.8 | 0.5 | 3.99 | 2.22 | 0.02 | 0.28 | 0.1 | 0.002 | 2.49 | 1.93 | 0.02 |
| AW | 1 year | 8.91 | 4.53 | | 2 | 1.68 | | 0.06 | 0.12 | | 1.29 | 1.03 | |

SCFA in feces

| Isovaleric Acid | | Valeric Acid | | Caproic acid | |
|---|---|---|---|---|---|
| Mean | SD | Mean | SD | Mean | SD |

| | | | | | |
|---|---|---|---|---|---|
| (μmol/g feces) | p | (μmol/g feces) | p | (μmol/g feces) | p |

| | | | | | | | | | |
|---|---|---|---|---|---|---|---|---|---|
| Controls | 0.13 | 0.17 | 0.48 | 0 | 0.01 | 0.1 | ND | — | — |
| AW | 0.18 | 0.23 | | 0.03 | 0.04 | | ND | — | |
| Controls | 0.41 | 0.14 | 0.02 | 0.21 | 0.18 | 0.007 | ND | — | — |
| AW | 0.22 | 0.22 | | 0.08 | 0.17 | | ND | — | |

SCFA in urine

| | | Mean | SD | | Mean | SD | | Mean | SD | | Mean | SD | |
|---|---|---|---|---|---|---|---|---|---|---|---|---|---|
| | | (μmol/mOsmol | | | (μmol/mOsmol | | | (μmol/mOsmol | | | (μmol/mOsmol | | |
| | Age | urine) | | p | urine) | | p | urine) | | p | urine) | | p |
| Controls | 3 mo | 1.4 | 1.28 | 0.74 | 0.23 | 0.24 | 0.82 | 0.05 | 0.17 | 0.42 | 0.17 | 0.11 | 0.39 |
| AW | 3 mo | 1.64 | 1.48 | | 0.37 | 0.5 | | 0.11 | 0.21 | | 0.29 | 1.4 | |
| Controls | 1 year | 1.25 | 0.32 | 0.72 | 0.14 | 0.09 | 0.15 | 0.01 | 0.03 | 0.17 | 0.1 | 0.14 | 0.8 |
| AW | 1 year | 1.19 | 0.6 | | 0.09 | 0.07 | | 0.03 | 0.03 | | 0.1 | 0.12 | |

SCFA in urine

| | Mean | SD | | Mean | SD | | Mean | SD | |
|---|---|---|---|---|---|---|---|---|---|
| | (μmol/mOsmol | | | (μmol/mOsmol | | | (μmol/mOsmol | | |
| | urine) | | p | urine) | | p | urine) | | p |
| Controls | 0.04 | 0.07 | 0.09 | 0.08 | 0.08 | 0.47 | 0.25 | 0.19 | 1 |
| AW | 0.23 | 0.37 | | 0.31 | 0.51 | | 0.48 | 0.63 | |
| Controls | 0.05 | 0.08 | 0.63 | 0.06 | 0.07 | 0.56 | 0.14 | 0.07 | 0.2 |
| AW | 0.06 | 0.07 | | 0.05 | 0.05 | | 0.11 | 0.07 | |

ND = not detected
SD = standard deviation

In animal models of asthma, propionate⁶, acetate⁶ and butyrate¹⁴ have all been shown to protect against airway inflammation, and this protective effect has been attributed to the stimulation of Tregs and dendritic cells capable of preventing Th2-type immune responses¹⁵.

Urine metabolomics analyses were used to identify metabolic differences of both host and microbial origin¹⁶. This technique has been used with high accuracy to discriminate between asthmatics and controls in humans¹⁷ and guinea pigs¹⁸. By comparing urine from AW and control subjects, a subtle yet significant metabolic signal was detected between the two phenotypes (of the 580 metabolites identified, 39 differed significantly at 3-months and 28 differed at 1-year). For the purpose of this paper we have focused on metabolites of microbial origin or contribution. Eight metabolites influenced by bacterial metabolism were differentially excreted in the urine of AW children compared to controls at 3-months of age, whereas only two were differentially detected at 1-year, reflecting once again the impact of microbial dysbiosis in early infancy. At 3-months, the excretion of sulphated bile acids glycolithocholate, glycocholenate and glycohyocholate was higher in AW children, while tauroursodeoxycholate excretion was decreased (FIG. 3B). This change in excretion could occur from an increase in host production of bile acids and/or a change in the microbial enzymatic activity on primary bile acids. Given the difference in several species of the gut microbial community in the AW children, it is likely that at least part of the difference in bile acid excretion is due to microbial dysbiosis¹⁹. Perhaps the most striking metabolic difference observed in the urine of AW children was the 14-fold increase in urobilinogen, a specific product of the gut microbiota. Urobilinogen is formed by the reduction of bilirubin, a breakdown product of heme catabolism. Clostridial species are the only known bacteria capable of bilirubin conversion^{20,21} and in this study Clostridium spp. decreased (NS) in abundance in the AW group at 3-months (FIG. 8), suggesting that the large elevation in urobilinogen excretion may be due instead to a change in host bilirubin metabolism. It is possible that the higher bile acid levels in the AW group led to an increase in bilirubin since erythrocytes are particularly susceptible to the membrane-damaging effect of bile acids²². Interestingly, unlike other bile acids, tauroursodeoxycholate has been shown to have a cytoprotective effect on erythrocytes and hepatocytes in the presence of other bile acids^{23,24} consistent with its higher level in the control group (FIG. 3B). The differences observed in bile acid metabolism correlate with an increase in urobilinogen excretion in urine of AW children at 3-months. It is unclear if and how these metabolic alterations are related to asthma pathogenesis. Nevertheless, they constitute a marker of gut dysbiosis in early infancy that can be detected in urine and that is linked to asthma risk.

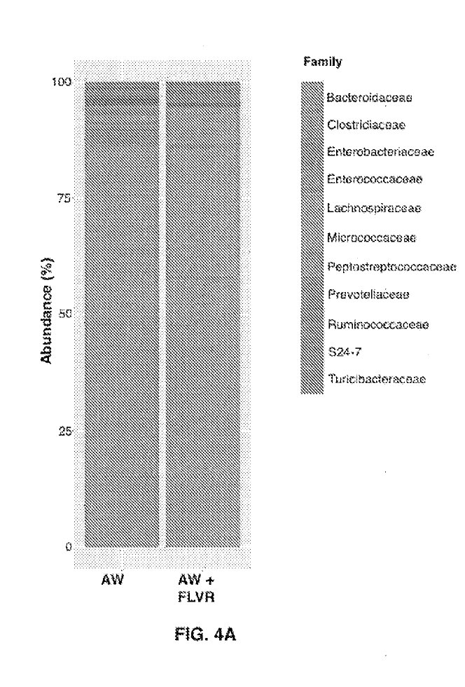

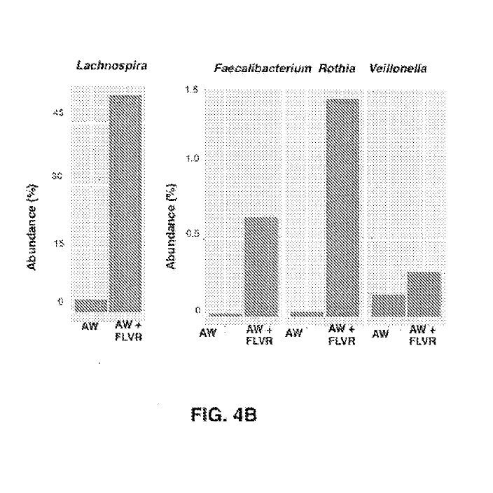

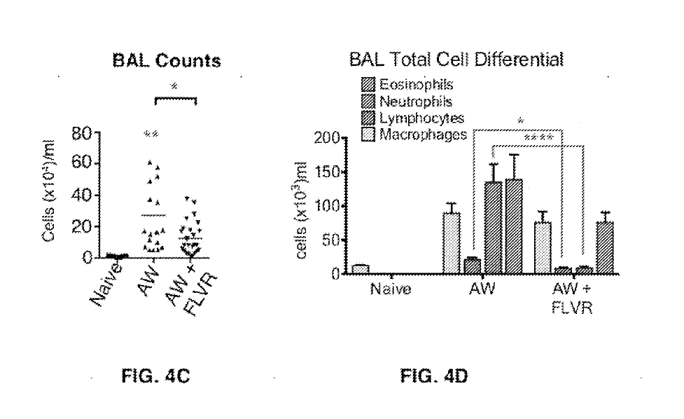

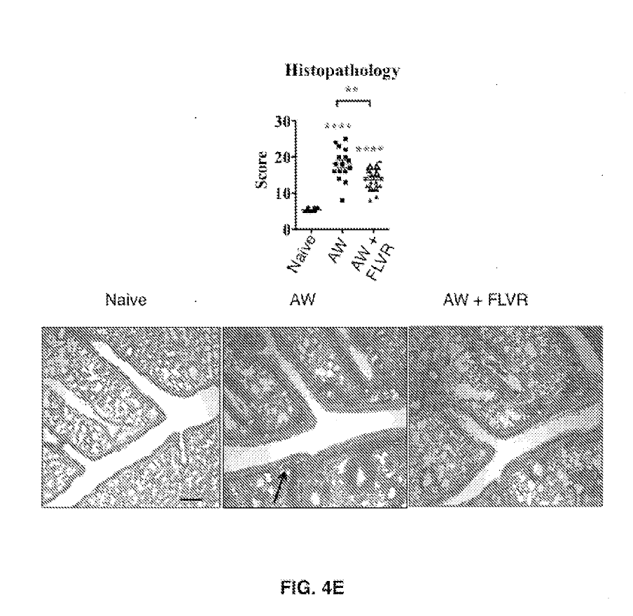

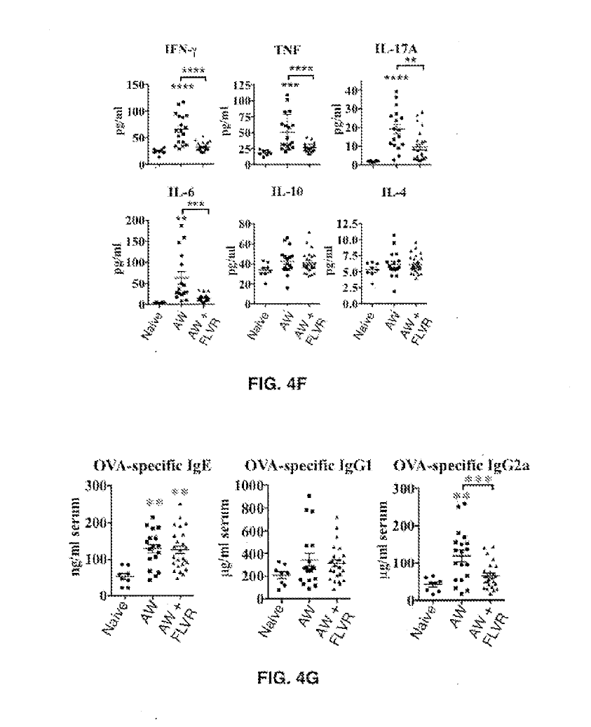

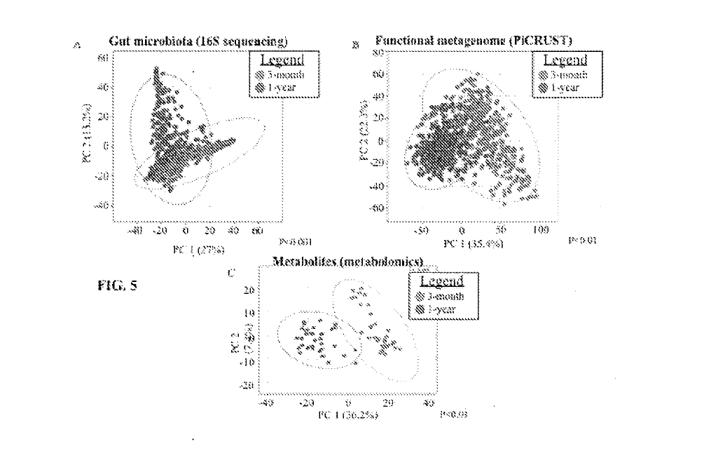

The role of Faecalibacterium sp., Lachnospira sp., Veillonella sp. and Rothia sp. (collectively abbreviated as FLVR) in asthma susceptibility was explored in a murine model of airway inflammation with humanized microbiota. Adult germ-free (GF) mice (n=4) were inoculated with feces from one AW subject collected at 3-months or with the same human inoculum deliberately supplemented with live FLVR. This AW subject was chosen based on the very low abundance of these 4 taxa in the 3-month feces, positive stringent API, and the formal diagnosis of asthma by 3-years of age. Mice born to parents harbouring FLVR successfully maintained these strains, with Lachnospira sp. colonizing at a much higher abundance than the other 3 strains (FIG. 4A, B). The F1 generation was immunized with ovalbumin (OVA) at 7-8 weeks of age to induce an airway inflammatory response. Mice inoculated with the AW microbiota exhibited a severe lung inflammatory response to OVA, characterized by a mixed lung infiltrate comprised of neutrophils, eosinophils, macrophages and lymphocytes. However, supplementation of the AW microbiota with FLVR significantly decreased the total lung cell infiltrate in the bronchoalveolar lavage (BAL; p<0.05) (FIG. 4C, D). Histopathological scoring confirmed that supplementation with FLVR reduced airway inflammation (FIG. 4E; p<0.01). In addition, FLVR supplementation significantly reduced the concentrations of key proinflammatory cytokines IFN-γ, TNF, IL-17A and IL-6 and OVA-specific IgG2a (FIG. 4F, G; p<0.01-0.0001). This cytokine pattern is reminiscent of the elevated TNF, IL-17A and IL-6 associated with severe human asthma with increased levels of neutrophils²⁵⁻²⁷. Together, these data show that the microbiota from the AW sample induced a mixed Th1/Th2/Th17 lung inflammatory response, and that deliberate, therapeutic colonization with FLVR significantly reduced the Th1/Th17 components of the immune response.

The present invention has been described with regard to one or more embodiments. However, it will be apparent to persons skilled in the art that a number of variations and modifications can be made without departing from the scope of the invention as defined in the claims.

All citations are hereby incorporated by reference.

### REFERENCES

1 World Health Organization: Asthma, <http://www.who.int/mediacentre/factsheets/fs307/en/>(2013).
2 Graham-Rowe, D. Lifestyle: when allergies go west. Nature 479, S2-S4 (2011).
3 Russell, S. L. et al. Early life antibiotic-driven changes in microbiota enhance susceptibility to allergic asthma. EMBO reports 13, 440-447, doi:10.1038/embor.2012.32 (2012).
4 Russell, S. L. et al. Perinatal antibiotic treatment affects murine microbiota, immune responses and allergic asthma. Gut microbes 4, 158-164, doi:10.4161/gmic.23567 (2013).
5 Arrieta, M. C., Stiems.ma, L. T., Amenyogbe, N., Brown, E. M. & Finlay, B. The intestinal microbiome in early life: health and disease. Front Immunol 5, 427, doi:10.3389/fimmu.2014.00427 (2014).
6 Trompette, A. et al. Gut microbiota metabolism of dietary fiber influences allergic airway disease and hematopoiesis. Nature medicine 20, 159-166, doi:10.1038/nm.3444 (2014).
7 Castro-Rodriguez, J. A., Holberg, C. J., Wright, A. L. & Martinez, F. D. A clinical index to define risk of asthma in young children with recurrent wheezing. American journal of respiratory and critical care medicine 162, 1403-1406, doi:10.1164/ajrccm.162.4.9912111 (2000).
8 Castro-Rodriguez, J. A., Cifuentes, L. & Rodriguez-Martinez, C. E. The asthma predictive index remains a useful tool to predict asthma in young children with recurrent wheeze in clinical practice. The Journal of allergy and clinical immunology 127, 1082-1083, doi:10.1016/jjaci.2011.01.024 (2011).
9 Hoskin-Parr, L., Teyhan, A., Blocker, A. & Henderson, A. J. Antibiotic exposure in the first two years of life and development of asthma and other allergic diseases by 7.5 yr: a dose-dependent relationship. Pediatric allergy and immunology : official publication of the European Society of Pediatric Allergy and Immunology 24, 762-771, doi:10.1111/pai.12153 (2013).
10 Azad, M. B. et al. Gut microbiota of healthy Canadian infants: profiles by mode of delivery and infant diet at 4 months. CMAJ: Canadian Medical Association journal=journal de l'Association medicale canadienne 185, 385-394, doi:10.1503/cmaj.121189 (2013).
11 Tanaka, S. et al. Influence of antibiotic exposure in the early postnatal period on the development of intestinal microbiota. FEMS immunology and medical microbiology 56, 80-87, doi:10.1111/j.1574-695X.2009.00553.x (2009).
12 Yatsunenko, T. et al. Human gut microbiome viewed across age and geography. Nature 486, 222-227, doi:10.1038/nature11053 (2012).
13 Abrahamsson, T. R. et al. Low gut microbiota diversity in early infancy precedes asthma at school age. Clinical and experimental allergy: journal of the British Society for Allergy and Clinical Immunology 44, 842-850, doi:10.1111/cea.12253 (2014).
14 Fallani, M. et al. Intestinal microbiota of 6-week-old infants across Europe: geographic influence beyond delivery mode, breast-feeding, and antibiotics. Journal of pediatric gastroenterology and nutrition 51, 77-84, doi:10.1097/MPG.0b013e3181d1b1 le (2010).
15 Arpaia, N. et al. Metabolites produced by commensal bacteria promote peripheral regulatory T-cell generation. Nature 504, 451-455, doi:10.1038/nature12726 (2013).
16 Holmes, E., Li, J. V., Athanasiou, T., Ashrafian, H. & Nicholson, J. K. Understanding the role of gut microbiome-host metabolic signal disruption in health and disease. Trends in microbiology 19, 349-359, doi:10.1016/j.tim.2011.05.006 (2011).
17 Saude, E. J. et al. Metabolomic profiling of asthma: diagnostic utility of urine nuclear magnetic resonance spectroscopy. The Journal of allergy and clinical immunology 127, 757-764 e751-756, doi:10.1016/j.jaci.2010.12.1077 (2011).
18 Saude, E. J. et al. Metabolomic biomarkers in a model of asthma exacerbation: urine nuclear magnetic resonance. American journal of respiratory and critical care medicine 179, 25-34, doi:10.1164/rccm.200711-17160C (2009).
19 Buffie, C. G. et al. Precision microbiome reconstitution restores bile acid mediated resistance to Clostridium difficile. Nature 517, 205-208, doi:10.1038/nature13828 (2015).
20 Midtvedt, T. & Gustafsson, B. E. Microbial conversion of bilirubin to urobilins in vitro and in vivo. Acta pathologica et microbiologica Scandinavica. Section B, Microbiology 89, 57-60 (1981).
21 Becker, N., Kunath, J., Loh, G. & Blaut, M. Human intestinal microbiota: characterization of a simplified and stable gnotobiotic rat model. Gut microbes 2, 25-33, doi:10.4161/gmic.2.1.14651 (2011).
22 Begley, M., Gahan, C. G. & Hill, C. The interaction between bacteria and bile. FEMS microbiology reviews 29, 625-651, doi:10.1016/j.femsre.2004.09.003 (2005).
23 Heuman, D. M., Pandak, W. M., Hylemon, P. B. & Vlahcevic, Z. R. Conjugates of ursodeoxycholate protect against cytotoxicity of more hydrophobic bile salts: in vitro studies in rat hepatocytes and human erythrocytes. Hepatology 14, 920-926 (1991).
24 Pazzi, P. et al. Bile salt-induced cytotoxicity and ursodeoxycholate cytoprotection: in-vitro study in perifused rat hepatocytes. European journal of gastroenterology & hepatology 9, 703-709 (1997).
25 Manise, M. et al. Sputum IgE and cytokines in asthma: relationship with sputum cellular profile. PloS one 8, e58388, doi:10.1371/joumal.pone.0058388 (2013).
26 Marini, M., Vittori, E., Hollemborg, J. & Mattoli, S. Expression of the potent inflammatory cytokines, granulocyte-macrophage-colony-stimulating factor and interleukin-6 and interleukin-8, in bronchial epithelial cells of patients with asthma. The Journal of allergy and clinical immunology 89, 1001-1009 (1992).
27 Virchow, J. C., Jr., Kroegel, C., Walker, C. & Matthys, H. Inflammatory determinants of asthma severity: mediator and cellular changes in bronchoalveolar lavage fluid of patients with severe asthma. The Journal of allergy and clinical immunology 98, S27-33; discussion S33-40 (1996).
28 Olszak, T. et al. Microbial exposure during early life has persistent effects on natural killer T cell function. Science 336, 489-493, doi:10.1126/science.1219328 (2012).
29. Hartmann et al., Significant and persistent impact of timber harvesting on soil microbial communities in Northern coniferous forests, The ISME Journal 6, 2199-2218 (2012).
30. DeSantis, T. Z. et al., Greengenes, a Chimera-Checked 16S rRNA Gene Database and Workbench Compatible with ARB, Appl. Environ. Microbiol. 72: 5069-5072 (2006).
31. Claesson et al, Comparison of two next-generation sequencing technologies for resolving highly complex microbiota composition using tandem variable 16S rRNA gene regions. Nucleic Acids Res 38:e200 (2010).
32. Konstantinidis et al, The bacterial species definition in the genomic era. Philos Trans R Soc Lond B Biol Sci 361: 1929-1940 (2006).
33. Achtman and Wagner. Microbial diversity and the genetic nature of microbial species. Nat. Rev. Microbiol. 6: 431-440 (2008).
34. Ausubel et al., Current Protocols in Molecular Biology, John Wiley & Sons, New York, N.Y. (2010).
35. Gennaro “Remington: The Science and Practice of Pharmacy,” 20^{th} Edition, Lippincott, Williams & Wilkins (ISBN: 0683306472) (2000).

1-24. (canceled)

25. A method of treating one or more of gut dysbiosis, asthma, allergy, atopic dermatitis, or atopy, or of altering the gut microbiota, or for populating the gastrointestinal tract, in a subject in need thereof, comprising administering to the subject an effective amount of a bacterial composition comprising bacteria of the genera Faecalibacterium and Lachnospira.

26. The method of claim 25, wherein the bacterial composition further comprises bacteria of the genus Veillonella.

27. The method of claim 25, wherein the bacterial composition further comprises bacteria of the genus Rothia.

28. The method of claim 25, wherein the bacterial composition further comprises bacteria of the genera Veillonella and Rothia.

29. The method of claim 25, wherein the subject is undergoing, will undergo, or has undergone antibiotic therapy.

30. The method of claim 25, wherein the subject is a human infant or a pregnant female.

31. The method of claim 30, wherein the human infant is less than one year old.

32. The method of claim 25, wherein the bacterial composition comprises at least one of the following species: Faecalibacterium prausnitzii, Faecalibacterium sp. CAG: 1138; Faecalibacterium sp. CAG:82; Faecalibacterium sp. CAG:74; Faecalibacterium sp. DJF VR20; Faecalibacterium sp. canine oral taxon 147; Faecalibacterium sp. MC 41, and any OTU encompassing at least one of said species.

33. The method of claim 32, wherein the bacterial composition comprises Faecalibacterium prausnitzii.

34. The method of claim 33, wherein the bacterial composition comprises at least one of Faecalibacterium prausnitzii L2-6, Faecalibacterium cf. prausnitzii KLE1255, Faecalibacterium prausnitzii SL3/3, Faecalibacterium prausnitzii M21/2, or Faecalibacterium prausnitzii A2-165, the Faecalibacterium prausnitzii deposited with the American Type Culture Collection (ATCC) under ATCC 27766 or ATCC 27768, a Faecalibacterium prausnitzii having a 16S rRNA gene having at least 95%, 96%, 97%, 98% or 99% sequence identity to the 16S rRNA gene set forth under GenBank accession number X85022.1 (SEQ ID NO: 1) or AJ413954.1 (SEQ ID NO: 2).

35. The method of claim 25, wherein the bacterial composition comprises at least one of the following species: Lachnospira multipara, Lachnospira pectinoschiza, or any operational taxonomic unit (OTU) encompassing at least one of said species.

36. The method of claim 35, wherein the bacterial composition comprises Lachnospira multipara.

37. The method of claim 36, wherein the bacterial composition comprises at least one of Lachnospira multipara D32, Lachnospira multipara LB2003, Lachnospira multipara MC2003, Lachnospira multipara DSM-3073, Lachnospira multipara deposited with the ATCC under ATCC 19207 or DSM-3073, a Lachnospira multipara DSM-3073 having at least 95%, 96%, 97%, 98% or 99% sequence identity to the 16S rRNA gene set forth under GenBank accession number FR733699.1 (SEQ ID NO: 3), Lachnospira pectinoschiza, strain 150-1, or Lachnospira pectinoschiza deposited with the ATCC under ATCC 49827.

38. The method of claim 33, wherein the bacterial composition comprises Lachnospira multipara.

39. The method of claim 26, wherein the bacterial composition comprises at least one of the following species: Veillonella parvula, Veillonella atypica, Veillonella parvula parvula, Veillonella dispar, Veillonella rogosae, Veillonella seninalis, Veillonella sp. oral taxon 780 str. F0422, Veillonella tobetsuensis, Veillonella montpellierensis, Veillonella magna, Veillonella sp. oral taxon 158 str. F0412, Veillonella ratti, Veillonella criceti, or any operational taxonomic unit (OTU) encompassing at least one of said species.

40. The method of claim 27, wherein the bacterial composition comprises at least one of the following species: Rothia mucilaginosa, Rothia dentocariosa, Rothia aeria, Rothia nasimurium, Rothia marina, Rothia terrae, Rothia endophytica, Rothia amarae, Rothia arfidiae, Rothia sp. CCUG 25688, Rothia sp. ChDC B201, Rothia sp. oral clone BP1-65, or any operational taxonomic unit (OTU) encompassing at least one of said species.

41. A method of treating one or more of gut dysbiosis, asthma, allergy, atopic dermatitis, or atopy, or of altering the gut microbiota, or for populating the gastrointestinal tract, in a subject in need thereof, comprising:
determining from a sample whether the subject has a decreased level of bacteria of the genera Faecalibacterium and Lachnospira relative to a reference or a healthy subject, and
if it is determined that the subject has a decreased level of bacteria of the genera Faecalibacterium and Lachnospira relative to a reference or a healthy subject, then administering to the subject an effective amount of a bacterial composition comprising bacteria of the genera Faecalibacterium and Lachnospira.

42. The method of claim 41, wherein the bacterial composition further comprises bacteria of the genus Veillonella.

43. The method of claim 41, wherein the bacterial composition further comprises bacteria of the genus Rothia.

44. The method of claim 41, wherein the bacterial composition further comprises bacteria of the genera Veillonella and Rothia.


SEQUENCE LISTING (91 sequences)

SEQ ID NO: 1 - DNA, 1499 bp, Faecalibacterium praussnitzii
  misc_feature (505)..(505): n is a, c, g, or t
  misc_feature (559)..(559): n is a, c, g, or t
  misc_feature (1026)..(1026): n is a, c, g, or t
  misc_feature (1097)..(1097): n is a, c, g, or t
gagagtttgattctggctcaggacgaacgctggcggcgcgcctaacacatgcaagtcgaacgagcgagagaggagcttgctttctcaagcgagtggcgaacgggtgagtaacgcgtgaggaacctgcctcaaagagggggacaacagttggaaacgactgctaataccgcataagcccacgacctggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaanacgtaggtcacaagcgttgtccggaattactgggtgtaaagggagcgcaggcgngaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcgcatagaaatatgtgtttcttcgggaccagagacaggtggtgcatggntgtcgtcagctcgtgtcgtgagatgttgggttaagtcccgcaacgagcgcaacccttatggtcagttactncgcaagaggactctggccagactgccgttgacaaaacggaggaaggtggggatgacgtcaaatcatcatgccctttatgacttgggctacacacgtactacaatggcgttaaacaaagagaagcaagaccgcgaggcgagcaaaactcagaaacttcgtcccagttcggactgcaggctgcaactcgcctgcacgaagtcggaattgctagtaatcgcagatcagcatgctgcggtgaatacgttcccgggcctgtacacaccgcccgtcacaccatgagagccggggggacccgaagtcggtagtctaaccgcaaggaggacgccgccgaagtaaaactggtgattggggtgaagtcgtaacaaggtagccgtagagaacctgcggctggatcacctcctt

SEQ ID NO: 2 - DNA, 1462 bp, Faecalibacterium praussnitzii
gatcctggctcaggcgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtac

SEQ ID NO: 3 - DNA, 1518 bp, Lachnospira multipara
tttgatcctggctcaggatgaacgctggcggcgtgcttaacacatgcaagtcgaacgaagcaacwtatcacgattccttcgggatgacgatttgttgactgagtggcggacgggtgagtaacgcgtgggtaacctgccttatacagggggatagcagctggaaacggctgataataccgcataagcgcacggcatcgcatgatgcagtgtgaaaaactccggtggtataagatggacccgcgtctgattagctagttggtgaggtaacggcccaccaaggcaacgatcagtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggagtgtaggtggcaaagcaagtcagtagtgaaaatccggggctcaacctcggaactgctattgaaactgtttagctagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactaggtgttgggtctcataagagattcggtgccgcagctaacgcaataagtattccacctggggagtacgttcgcaagaatgaaactcaaaggaattgacggggacccgcacaagcggtggagcatgtggtttaattcgaagcaacgcgaagaaccttacctagtcttgacatcccgatgaccragtatgtaatgtactctttcttcggaacatcggtgacaggtggtgcatggttgtcgtcagctcgtgtcgtgagatgttgggttaagtcccgcaacgagcgcaacccctatttctagtagccagcagttcggctgggcactctagagagactgccagggataacctggaggaaggtggggatgacgtcaaatcatcatgccccttatgactagggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcct

SEQ ID NO: 4 - DNA, 1497 bp, Veillonella parvula
gctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtyagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaacyagagatrgttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagcc

SEQ ID NO: 5 - DNA, 1467 bp, Rothia mucilaginosa
ctggctcaggacgaacgctggcggcgtgcttaacacatgcaagtcgaacgatgaaccctagcttgctaggtggattagtggcgaacgggtgagtaacacgtgagtaacctacccttaactctgggataagcctgggaaactgggtctaataccggatacgaccaatctccgcatggggtgttggtggaaagcgttatgtagtggttatagatgggctcacggcctatcagcttgttggtgaggtaacggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcagggaagaagagagattgacggtacctgcagagaaagcgccggctaactacgtgccagcagccgcggtaatacgtagggcgcgagcgttgtccggaattattgggcgtaaagagcttgtaggcggtttgtcgcgtctgctgtgaaaggccggggcttaaccccgtgtattgcagtgggtacgggcagactagagtgcagtaggggagactggaattcctggtgtagcggtggaatgcgcagatatcaggaagaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggaccgcatcagagatggtgtttcccttcggggctggtatacaggtggtgcatggttgtcgtcagctcgtgtcgtgagatgttgggttaagtcccgcaacgagcgcaaccctcgttctatgttgccagcacgttatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccaaagccggtggcctaacctcttttgggagggagccgtctaaggtgggatcggcgattgggactaagtcg

SEQ ID NO: 6 - DNA, 21 bp, Artificial sequence
  Forward primer for all bacteria
actcctacgggaggcagcagt

SEQ ID NO: 7 - DNA, 18 bp, Artificial sequence
  Reverse primer for all bacteria
attaccgcggctgctggc

SEQ ID NO: 8 - DNA, 21 bp, Artificial sequence
  Forward primer for Bifidobacterium sp.
ctcctggaaacgggtggtaat

SEQ ID NO: 9 - DNA, 21 bp, Artificial sequence
  Reverse primer for Bifidobacterium sp.
ataggacgcgaccccatccca

SEQ ID NO: 10 - DNA, 20 bp, Artificial sequence
  Forward primer for Veillonella sp.
aagctatcactgaaggaggg

SEQ ID NO: 11 - DNA, 21 bp, Artificial sequence
  Reverse primer for Veillonella sp.
tcccaatgtggccgttcatcc

SEQ ID NO: 12 - DNA, 21 bp, Artificial sequence
  Forward primer for Rothia sp.
gcctgggaaactgggtctaat

SEQ ID NO: 13 - DNA, 19 bp, Artificial sequence
  Reverse primer for Rothia sp.
caagctgataggccgtgag

SEQ ID NO: 14 - DNA, 21 bp, Artificial sequence
  Forward primer for Faecalibacterium sp.
ggagcgatccgctttgagatg

SEQ ID NO: 15 - DNA, 22 bp, Artificial sequence
  Reverse primer for Faecalibacterium sp.
aacctctcagtccggctaccga

SEQ ID NO: 16 - DNA, 20 bp, Artificial sequence
  Forward primer for Lachnospira sp.
gcaacgcgaagaaccttacc

SEQ ID NO: 17 - DNA, 20 bp, Artificial sequence
  Reverse primer for Lachnospira sp.
accacctgtcaccgatgttc

SEQ ID NO: 18 - DNA, 27 bp, Artificial sequence
  16S V3 region forward primer
  misc_feature (7)..(10): n is a, c, g, or t
ctgatcnnnncctacgggaggcagcag

SEQ ID NO: 19 - DNA, 23 bp, Artificial sequence
  16S V3 region reverse primer
aaccccattaccgcggctgctgg

SEQ ID NO: 20 - DNA, 27 bp, Artificial sequence
  16S V3 region forward primer
  misc_feature (7)..(10): n is a, c, g, or t
agcatcnnnncctacgggaggcagcag

SEQ ID NO: 21 - DNA, 23 bp, Artificial sequence
  16S V3 region reverse primer
ccaacaattaccgcggctgctgg

SEQ ID NO: 22 - DNA, 27 bp, Artificial sequence
  16S V3 region forward primer
  misc_feature (7)..(10): n is a, c, g, or t
cgattannnncctacgggaggcagcag

SEQ ID NO: 23 - DNA, 23 bp, Artificial sequence
  16S V3 region reverse primer
agttccattaccgcggctgctgg

SEQ ID NO: 24 - DNA, 27 bp, Artificial sequence
  16S V3 region forward primer
  misc_feature (7)..(10): n is a, c, g, or t
cattcannnncctacgggaggcagcag

SEQ ID NO: 25 - DNA, 23 bp, Artificial sequence
  16S V3 region reverse primer
accggcattaccgcggctgctgg

SEQ ID NO: 26 - DNA, 27 bp, Artificial sequence
  16S V3 region forward primer
  misc_feature (7)..(10): n is a, c, g, or t
aagctannnncctacgggaggcagcag

SEQ ID NO: 27 - DNA, 23 bp, Artificial sequence
  16S V3 region reverse primer
caactaattaccgcggctgctgg

SEQ ID NO: 28 - DNA, 27 bp, Artificial sequence
  16S V3 region forward primer
  misc_feature (7)..(10): n is a, c, g, or t
gctgtannnncctacgggaggcagcag

SEQ ID NO: 29 - DNA, 23 bp, Artificial sequence
  16S V3 region reverse primer
ccacgcattaccgcggctgctgg

SEQ ID NO: 30 - DNA, 27 bp, Artificial sequence
  16S V3 region forward primer
  misc_feature (7)..(10): n is a, c, g, or t
atggcannnncctacgggaggcagcag

SEQ ID NO: 31 - DNA, 23 bp, Artificial sequence
  16S V3 region reverse  primer
ctatacattaccgcggctgctgg

SEQ ID NO: 32 - DNA, 27 bp, Artificial sequence
  16S V3 region forward primer
  misc_feature (7)..(10): n is a, c, g, or t
gcctaannnncctacgggaggcagcag

SEQ ID NO: 33 - DNA, 23 bp, Artificial sequence
  16S V3 region reverse  primer
tacagcattaccgcggctgctgg

SEQ ID NO: 34 - DNA, 27 bp, Artificial sequence
  16S V3 region forward primer
  misc_feature (7)..(10): n is a, c, g, or t
gtagccnnnncctacgggaggcagcag

SEQ ID NO: 35 - DNA, 23 bp, Artificial sequence
  16S V3 region reverse primer
atgtcaattaccgcggctgctgg

SEQ ID NO: 36 - DNA, 27 bp, Artificial sequence
  16S V3 region forward primer
  misc_feature (7)..(10): n is a, c, g, or t
aagtgcnnnncctacgggaggcagcag

SEQ ID NO: 37 - DNA, 23 bp, Artificial sequence
  16S V3 region reverse primer
ttaggcattaccgcggctgctgg

SEQ ID NO: 38 - DNA, 27 bp, Artificial sequence
  16S V3 region forward primer
  misc_feature (7)..(10): n is a, c, g, or t
attatannnncctacgggaggcagcag

SEQ ID NO: 39 - DNA, 23 bp, Artificial sequence
  16S V3 region reverse primer
ggctacattaccgcggctgctgg

SEQ ID NO: 40 - DNA, 27 bp, Artificial sequence
  16S V3 region forward primer
  misc_feature (7)..(10): n is a, c, g, or t
ccagcannnncctacgggaggcagcag

SEQ ID NO: 41 - DNA, 23 bp, Artificial sequence
  16S V3 region reverse primer
acgataattaccgcggctgctgg

SEQ ID NO: 42 - DNA, 27 bp, Artificial sequence
  16S V3 region forward primer
  misc_feature (7)..(10): n is a, c, g, or t
tggtcannnncctacgggaggcagcag

SEQ ID NO: 43 - DNA, 23 bp, Artificial sequence
  16S V3 region reverse primer
ctcagaattaccgcggctgctgg

SEQ ID NO: 44 - DNA, 27 bp, Artificial sequence
  16S V3 region forward primer
  misc_feature (7)..(10): n is a, c, g, or t
ccactcnnnncctacgggaggcagcag

SEQ ID NO: 45 - DNA, 23 bp, Artificial sequence
  16S V3 region reverse primer
ccgtccattaccgcggctgctgg

SEQ ID NO: 46 - DNA, 27 bp, Artificial sequence
  16S V3 region forward primer
  misc_feature (7)..(10): n is a, c, g, or t
cgcggcnnnncctacgggaggcagcag

SEQ ID NO: 47 - DNA, 23 bp, Artificial sequence
  16S V3 region reverse primer
tgaccaattaccgcggctgctgg

SEQ ID NO: 48 - DNA, 27 bp, Artificial sequence
  16S V3 region forward primer
  misc_feature (7)..(10): n is a, c, g, or t
gaatgannnncctacgggaggcagcag

SEQ ID NO: 49 - DNA, 23 bp, Artificial sequence
  16S V3 region reverse primer
cttgtaattaccgcggctgctgg

SEQ ID NO: 50 - DNA, 27 bp, Artificial sequence
  16S V3 region forward primer
  misc_feature (7)..(10): n is a, c, g, or t
gcgccannnncctacgggaggcagcag

SEQ ID NO: 51 - DNA, 23 bp, Artificial sequence
  16S V3 region reverse primer
aagcgaattaccgcggctgctgg

SEQ ID NO: 52 - DNA, 27 bp, Artificial sequence
  16S V3 region forward primer
  misc_feature (7)..(10): n is a, c, g, or t
ctctacnnnncctacgggaggcagcag

SEQ ID NO: 53 - DNA, 23 bp, Artificial sequence
  16S V3 region reverse primer
tcattcattaccgcggctgctgg

SEQ ID NO: 54 - DNA, 27 bp, Artificial sequence
  16S V3 region forward primer
  misc_feature (7)..(10): n is a, c, g, or t
ggtttcnnnncctacgggaggcagcag

SEQ ID NO: 55 - DNA, 23 bp, Artificial sequence
  16S V3 region reverse primer
tggcgcattaccgcggctgctgg

SEQ ID NO: 56 - DNA, 27 bp, Artificial sequence
  16S V3 region forward primer
  misc_feature (7)..(10): n is a, c, g, or t
taaggcnnnncctacgggaggcagcag

SEQ ID NO: 57 - DNA, 23 bp, Artificial sequence
  16S V3 region reverse primer
aaggacattaccgcggctgctgg

SEQ ID NO: 58 - DNA, 27 bp, Artificial sequence
  16S V3 region forward primer
  misc_feature (7)..(10): n is a, c, g, or t
tcgggannnncctacgggaggcagcag

SEQ ID NO: 59 - DNA, 23 bp, Artificial sequence
  16S V3 region reverse primer
atcctaattaccgcggctgctgg

SEQ ID NO: 60 - DNA, 27 bp, Artificial sequence
  16S V3 region forward primer
  misc_feature (7)..(10): n is a, c, g, or t
ttcgaannnncctacgggaggcagcag

SEQ ID NO: 61 - DNA, 23 bp, Artificial sequence
  16S V3 region reverse primer
cactcaattaccgcggctgctgg

SEQ ID NO: 62 - DNA, 27 bp, Artificial sequence
  16S V3 region forward primer
  misc_feature (7)..(10): n is a, c, g, or t
gcggacnnnncctacgggaggcagcag

SEQ ID NO: 63 - DNA, 23 bp, Artificial sequence
  16S V3 region reverse primer
ccgcaaattaccgcggctgctgg

SEQ ID NO: 64 - DNA, 27 bp, Artificial sequence
  16S V3 region forward primer
  misc_feature (7)..(10): n is a, c, g, or t
attggcnnnncctacgggaggcagcag

SEQ ID NO: 65 - DNA, 23 bp, Artificial sequence
  16S V3 region reverse primer
gaaaccattaccgcggctgctgg

SEQ ID NO: 66 - DNA, 27 bp, Artificial sequence
  16S V3 region forward primer
  misc_feature (7)..(10): n is a, c, g, or t
ttattcnnnncctacgggaggcagcag

SEQ ID NO: 67 - DNA, 23 bp, Artificial sequence
  16S V3 region reverse primer
gccttaattaccgcggctgctgg

SEQ ID NO: 68 - DNA, 27 bp, Artificial sequence
  16S V3 region forward primer
  misc_feature (7)..(10): n is a, c, g, or t
tggagcnnnncctacgggaggcagcag

SEQ ID NO: 69 - DNA, 23 bp, Artificial sequence
  16S V3 region reverse primer
tcccgaattaccgcggctgctgg

SEQ ID NO: 70 - DNA, 27 bp, Artificial sequence
  16S V3 region forward primer
  misc_feature (7)..(10): n is a, c, g, or t
cttcgannnncctacgggaggcagcag

SEQ ID NO: 71 - DNA, 23 bp, Artificial sequence
  16S V3 region reverse primer
ttcgaaattaccgcggctgctgg

SEQ ID NO: 72 - DNA, 27 bp, Artificial sequence
  16S V3 region forward primer
  misc_feature (7)..(10): n is a, c, g, or t
ggagaannnncctacgggaggcagcag

SEQ ID NO: 73 - DNA, 23 bp, Artificial sequence
  16S V3 region reverse primer
gtccgcattaccgcggctgctgg

SEQ ID NO: 74 - DNA, 27 bp, Artificial sequence
  16S V3 region forward primer
  misc_feature (7)..(10): n is a, c, g, or t
tttcacnnnncctacgggaggcagcag

SEQ ID NO: 75 - DNA, 23 bp, Artificial sequence
  16S V3 region reverse primer
aaagcaattaccgcggctgctgg

SEQ ID NO: 76 - DNA, 27 bp, Artificial sequence
  16S V3 region forward primer
  misc_feature (7)..(10): n is a, c, g, or t
tccgtcnnnncctacgggaggcagcag

SEQ ID NO: 77 - DNA, 23 bp, Artificial sequence
  16S V3 region reverse primer
agaagaattaccgcggctgctgg

SEQ ID NO: 78 - DNA, 27 bp, Artificial sequence
  16S V3 region forward primer
  misc_feature (7)..(10): n is a, c, g, or t
tgtgccnnnncctacgggaggcagcag

SEQ ID NO: 79 - DNA, 23 bp, Artificial sequence
  16S V3 region reverse primer
gaataaattaccgcggctgctgg

SEQ ID NO: 80 - DNA, 27 bp, Artificial sequence
  16S V3 region forward primer
  misc_feature (7)..(10): n is a, c, g, or t
tgccgannnncctacgggaggcagcag

SEQ ID NO: 81 - DNA, 23 bp, Artificial sequence
  16S V3 region reverse primer
gctccaattaccgcggctgctgg

SEQ ID NO: 82 - DNA, 27 bp, Artificial sequence
  16S V3 region forward primer
  misc_feature (7)..(10): n is a, c, g, or t
ggccacnnnncctacgggaggcagcag

SEQ ID NO: 83 - DNA, 23 bp, Artificial sequence
  16S V3 region reverse primer
ttctccattaccgcggctgctgg

SEQ ID NO: 84 - DNA, 27 bp, Artificial sequence
  16S V3 region forward primer
  misc_feature (7)..(10): n is a, c, g, or t
tatatcnnnncctacgggaggcagcag

SEQ ID NO: 85 - DNA, 23 bp, Artificial sequence
  16S V3 region reverse primer
gtgaaaattaccgcggctgctgg

SEQ ID NO: 86 - DNA, 27 bp, Artificial sequence
  16S V3 region forward primer
  misc_feature (7)..(10): n is a, c, g, or t
caggccnnnncctacgggaggcagcag

SEQ ID NO: 87 - DNA, 23 bp, Artificial sequence
  16S V3 region reverse primer
cagatcattaccgcggctgctgg

SEQ ID NO: 88 - DNA, 27 bp, Artificial sequence
  16S V3 region reverse primer
  misc_feature (7)..(10): n is a, c, g, or t
ggtagannnncctacgggaggcagcag

SEQ ID NO: 89 - DNA, 23 bp, Artificial sequence
  16S V3 region reverse primer
aaatgcattaccgcggctgctgg

SEQ ID NO: 90 - DNA, 27 bp, Artificial sequence
  16S V3 region forward primer
  misc_feature (7)..(10): n is a, c, g, or t
cgaaacnnnncctacgggaggcagcag

SEQ ID NO: 91 - DNA, 23 bp, Artificial sequence
  16S V3 region reverse primer
acaaacattaccgcggctgctgg